SEQUENCE LISTING (9 sequences)

SEQ ID NO: 1 - protein, 262 aa, Escherichia coli (strain K12)
MQVDLLGSAQSAHALHLFHQHSPLVHCMTNDVVQTFTANTLLALGASPAMVIETEEASQFAAIASALLINVGTLTQPRAQAMRAAVEQAKSSQTPWTLDPVAVGALDYRRHFCHELLSFKPAAIRGNASEIMALAGIANGGRGVDTTDAAANAIPAAQTLARETGAIVVVTGEMDYVTDGHRIIGIHGGDPLMTKVVGTGCALSAVVAACCALPGDTLENVASACHWMKQAGERAVARSEGPGSFVPHFLDALWQLTQEVQA

SEQ ID NO: 2 - protein, 272 aa, Bacillus subtilis
MDAQSAAKCLTAVRRHSPLVHSITNNVVTNFTANGLLALGASPVMAYAKEEVADMAKIAGALVLNIGTLSKESVEAMIIAGKSANEHGVPVILDPVGAGATPFRTESARDIIREVRLAAIRGNAAEIAHTVGVTDWLIKGVDAGEGGGDIIRLAQQAAQKLNTVIAITGEVDVIADTSHVYTLHNGHKLLTKVTGAGCLLTSVVGAFCAVEENPLFAAIAAISSYGVAAQLAAQQTADKGPGSFQIELLNKLSTVTEQDVQEWATIERVTVS

SEQ ID NO: 3 - protein, 267 aa, Rhizobium leguminosarum bv. viciae (strain 3841)
MQTRTTPGAMLKAMREKPPLVQCITNYVAMNIAANVLLAAGASPAMVHAAEEAGEFAAIASALTINIGTLSTQWIDGMQAAAKAATSAGKPWVLDPVAHYATAFRRNAVAELLALKPTIIRGNASEIIALAGGESRGQGVDSRDPVEQAEGSARWLAERQRAVVAVTGAVDFVTDGERAVRIEGGSALMPQVTALGCSLTCLVGAFAATAPEDIFGATVAALSTFAIAGEEAALGAAGPGSFSWRFLDALAALDAETLDARARISAA

SEQ ID NO: 4 - protein, 602 aa, Populus trichocarpa
MATELLCLHRPISLTHKLFRNPLPKVIQATPLTLKLRCSVSTENVSFTETETETRRSANYEPNSWDYDYLLSSDTDESRKGRSSVIEVYKDKAKKLEAEVRREINNEKAEFLTLLELIDNVQRLGLGYRFESDIRRALDRFVSSGGFDAVTKTSLHATALSFRLLRQHGFEVSQEAFGGFKDQNGNFMENLKEDIKAILSLYEASFLALEGENILDEAKVFAISHLKELSEEKIGKDLAEQVNHALELPLHRRTQRLEAVLSIEAYRKKEDADQVLLELAILDYNMIQSVYQRDLRETSRWWRRVGLATKLHFARDRLIESFYWAVGVAFEPQYSDCRNSVAKMFSFVTIIDDIYDVYGTLDELELFTNAVERWDVNAIDDLPDYMKLCFLALYNTINEIAYDNLKEKGENILPYLTKAWADLCNAFLQEAKWLYNKSTPTFDDYFGNAWKSSSGPLQLVFAYFAVVQNIKKEEIENLKKYHDIISRPSHIFRLCNDLASASAEIARGETANSVSCYMRTKGISEELATESVMNLIDETWKKMNKEKLGGSLFAKPFVETAINLARQSHCTYHNGDAHTSPDELTRKRVLSVITEPILPFER

SEQ ID NO: 5 - protein, 608 aa, Pueraria lobata var. montanta
MATNLLCLSNKLSSPTPTPSTRFPQSKNFITQKTSLANPKPWRVICATSSQFTQITEHNSRRSANYQPNLWNFEFLQSLENDLKVEKLEEKATKLEEEVRCMINRVDTQPLSLLELIDDVQRLGLTYKFEKDIIKALENIVLLDENKKNKSDLHATALSFRLLRQHGFEVSQDVFERFKDKEGGFSGELKGDVQGLLSLYEASYLGFEGENLLEEARTFSITHLKNNLKEGINTKVAEQVSHALELPYHQRLHRLEARWFLDKYEPKEPHHQLLLELAKLDFNMVQTLHQKELQDLSRWWTEMGLASKLDFVRDRLMEVYFWALGMAPDPQFGECRKAVTKMFGLVTIIDDVYDVYGTLDELQLFTDAVERWDVNAINTLPDYMKLCFLALYNTVNDTSYSILKEKGHNNLSYLTKSWRELCKAFLQEAKWSNNKIIPAFSKYLENASVSSSGVALLAPSYFSVCQQQEDISDHALRSLTDFHGLVRSSCVIFRLCNDLATSAAELERGETTNSIISYMHENDGTSEEQAREELRKLIDAEWKKMNRERVSDSTLLPKAFMEIAVNMARVSHCTYQYGDGLGRPDYATENRIKLLLIDPFPINQLMYV

SEQ ID NO: 6 - protein, 582 aa, Eucalyptus globulus
MALRLLFTPHLPVLSSRRANGRVRCSASTQISDPQEGRRSANYQPSVWTYNYLQSIVAGEGRQSRREVEQQKEKVQILEEEVRGALNDEKAETFTIFATVDDIQRLGLGDHFEEDISNALRRCVSKGAVFMSLQKSLHGTALGFRLLRQHGYEVSQDVFKIFLDESGSFVKTLGGDVQGVLSLYEASHLAFEEEDILHKARSFAIKHLENLNSDVDKDLQDQVKHELELPLHRRMPLLEARRSIEAYSRREYTNPQILELALTDFNVSQSTLQRDLQEMLGWWNNTGLAKRLSFARDRLIECFFWAVGIAHEPSLSICRKAVTKAFALILVLDDVYDVFGTLEELELFTEAVRRWDLNAVEDLPVYMKLCYLALYNSVNEMAYETLKEKGENVIPYLAKAWYDLCKAFLQEAKWSNSRIIPGVEEYLNNGWVSSSGSVMLIHAYFLASPSIRKEELESLEHYHDLLRLPSLIFRLTNDIASSSAELERGETTNSIRCFMQEKGISELEARECVKEEIDTAWKKMNKYMVDRSTFNQSFVRMTYNLARMAHCVYQDGDAIGSPDDLSWNRVHSLIIKPISPAA

SEQ ID NO: 7 - protein, 583 aa, Melaleuca alternifolia
MALRLLSTPHLPQLCSRRVSGRVHCSASTQVSDAQGGRRSANYQPSVWTYNYLQSLVADDIRRSRREVEQEREKAQILEEDVRGALNDGNAEPMAIFALVDDIQRLGLGRYFEEDISKALRRCLSQYAVTGSLQKSLHGTALSFRVLRQHGFEVSQDVFKIFMDESGSFMKTLGGDVQGMLSLYEASHLAFEEEDILHKAKTFAIKHLENLNHDIDQDLQDHVNHELELPLHRRMPLLEARRFIEAYSRRSNVNPRILELAVMKFNSSQLTLQRDLQDMLGWWNNVGLAKRLSFARDRLMECFFWAVGIAREPALSNCRKGVTKAFSLILVLDDVYDVFGTLDELELFTDAVRRWHEDAVENLPGYMKLCFLALYNSVNDMAYETLKETGENVTPYLTKVWYDLCKAFLQEAKWSYNKITPGVEEYLNNGWVSSSGQVMLTHAYFLSSPSLRKEELESLEHYHDLLRLPSLIFRLTNDLATSSAELGRGETTNSILCYMREKGFSESEARKQVIEQIDTAWRQMNKYMVDHSTFNRSFMQMTYNLARMAHCVYQDGDAIGAPDDQSWNRVHSLIIKPVSLAPC

SEQ ID NO: 8 - protein, 576 aa, Malus domestica
MEFRVHLQADNEQKIFQNQMKPEPEASYLINQRRSANYKPNIWKNDFLDQSLISKYDGDEYRKLSEKLIEEVKIYISAETMDLVAKLELIDSVRKLGLANLFEKEIKEALDSIAAIESDNLGTRDDLYGTALHFKILRQHGYKVSQDIFGRFMDEKGTLENHHFAHLKGMLELFEASNLGFEGEDILDEAKASLTLALRDSGHICYPDSNLSRDVVHSLELPSHRRVQWFDVKWQINAYEKDICRVNATLLELAKLNFNVVQAQLQKNLREASRWWANLGIADNLKFARDRLVECFACAVGVAFEPEHSSFRICLTKVINLVLIIDDVYDIYGSEEELKHFTNAVDRWDSRETEQLPECMKMCFQVLYNTTCEIAREIEEENGWNQVLPQLTKVWADFCKALLVEAEWYNKSHIPTLEEYLRNGCISSSVSVLLVHSFFSITHEGTKEMADFLHKNEDLLYNISLIVRLNNDLGTSAAEQERGDSPSSIVCYMREVNASEETARKNIKGMIDNAWKKVNGKCFTTNQVPFLSSFMNNATNMARVAHSLYKDGDGFGDQEKGPRTHILSLLFQPLVN

SEQ ID NO: 9 - protein, 601 aa, Vitis vinifera
MALHLFYFPKQCFLTHNLPGHPMKKPPRGTTAQIRCSANEQSFSLMTESRRSAHYQPAFWSYDFVESLKKREEICDGSVKELEKMYEDRARKLEDEVKWMIHEKSAEPLTLLEFIDDIQRLGLGHRFENDIKRSLDKILLLEGSNAGKGESLHHTALRFRILKQHGYKVSQEVFEGFTDQNGHFKACLCKDVKGMLSLYEASYLASEGETLLHEAMAFLKMHLKDLEGTLDKSLEELVNHAMELPLHRRMPRLEARWFIEAYKRREGADDVLLELAILDFNMVQWTLQDDLQDMSRWWKDMGLASKLHFARDRLMECFFWTVGMAFEPEFSNCRKGLTKVTSFITTIDDVYDVYGSVDELELFTDAVARWDINMVNNLPGYMKLCFLALYNTVNEMAYDTLKEQGHNILPYLTKAWADLCKVFLVEAKWCHKEYTPTFEEYLENGWRSVSGAAILIHAYFLMSKNITKEALECLENDHELLRWPSTIFRLCNDLATSKAELERGESANSISCYMHQTGVSEEDAREHMKILIDESWKKMNKVREMDSDSPFAKPFVETAINLARIAQCTYQYGDSHGAPDARSKKRVLSLIVEPIPMNLKK

(19) United States
(12) Patent Application Publication
(10) Pub. No.: US 2015/0284743 A1
(43) Pub. Date: Oct. 8, 2015

(54) METHODS FOR THE ENZYMATIC PRODUCTION OF ISOPRENE FROM ISOPRENOL

(71) Applicants: GLOBAL BIOENERGIES, Evry Cedex (FR); SCIENTIST OF FORTUNE S.A., Luxembourg (LU)

(72) Inventors: Philippe Marliere, Tournai (BE); Maria Anissimova, Nozay (FR)

(73) Assignees: Global Bioenergies a corporation; Scientist of Fortune ,S.A. a corporation

(21) Appl. No.: 14/442,005

(22) PCT Filed: Nov. 8, 2013
(86) PCT No.: PCT/EP2013/073425

§ 371 Date: May 11, 2015

(30) Foreign Application Priority Data
Nov. 13, 2012 (EP) 12192428.6

(51) Int. Cl. C12P 5/00; C12N 9/10; C12N 9/90; C12N 9/12; C12N 9/88

(52) U.S. Cl. CPC C12P 5/007; C12N 9/1205; C12N 9/88; C12N 9/90; C12N 9/13; C12Y 207/0105; C12Y 208/02; C12Y 402/03014; C12Y 402/03015; C12Y 402/03046; C12Y 402/03047; C12Y 503/03002

(57) ABSTRACT

Described are methods for the enzymatic production of isoprene which allow to produce isoprene from isoprenol and microorganisms which have been genetically modified so as to be able to produce isoprene from isoprenol.

Also described are enzyme combinations which allow to convert isoprenol into isoprene as well as (micro)organisms which express such enzyme combinations.

### CROSS REFERENCE TO RELATED APPLICATIONS

This application is a 371 National Phase filing of PCT/EP2013/073425 filed Nov. 8, 2013, which is a continuation of EP 12 192 428 which was filed on Nov. 13, 2012, which are incorporated by reference in their entirety.

### FIELD OF THE INVENTION

The present invention relates to methods for the enzymatic production of isoprene which allow to produce isoprene from isoprenol. The present invention also relates to microorganisms which have been genetically modified so as to produce isoprene from isoprenol.

The present invention furthermore relates to enzyme combinations which allow to convert isoprenol into isoprene as well as to (micro)organisms which express such enzyme combinations.

### BACKGROUND OF THE INVENTION

Isoprene (2-methyl-1,3-butadiene; see FIG. 1) is a volatile hydrocarbon that is insoluble in water and soluble in alcohol. Commercially viable quantities of isoprene can be obtained by direct isolation from petroleum C5 cracking fractions or by dehydration of C5 isoalkanes or isoalkenes. The C5 skeleton can also be synthesised from smaller subunits. Due to the desire to be able to produce isoprene in methods which are independent from non-renewable resources, attempts have been made to provide methods for producing isoprene enzymatically making use of genetically modified microorganisms. In nature isoprene production occurs by two distinct metabolic pathways (Julsing et al.; Appl. Microbiol. Technol. 75 (2007), 1377-1384). In eukaryotes and archae isoprene is formed via the mevalonate (MVA) pathway, while some eubacteria and higher plants produce isoprene via the methylerythritol phosphate (MEP) pathway. Accordingly, there are some reports on the genetic modification of microorganisms exploiting these pathways. For example, WO2010/031062 describes the increase of isoprene production by using the archaeal lower mevalonate pathway. US 2011/0039323 A1 describes a method for producing isoprene by providing microorganisms that express certain enzymes of the MEP pathway. WO2010/031076 describes the conversion of prenyl derivatives into isoprene by making use of isoprene synthase. This includes the conversion of isoprenol diphosphate and prenol diphosphate into isoprene using an isoprene synthase. Although these processes provide some progress in the production of isoprene based on renewable resources, there is still a need to provide corresponding methods which allow a further improvement as regards efficiency of production.

### BRIEF SUMMARY OF THE INVENTION

The present invention addresses this need and provides for methods for the enzymatic production of isoprene which allow to produce isoprene from isoprenol.

Thus, in a first aspect the present invention relates to a method for the production of isoprene in which isoprenol is first enzymatically converted into isoprenyl monophosphate and in which isoprenyl monophosphate is then enzymatically further converted into isoprene according to the following general scheme (see also FIG. 2):

Isoprenol [symbol] isoprenyl monophosphate [symbol] isoprene

According to the present invention the conversion of isoprenol into isoprenyl monophosphate occurs preferably according to the following reaction:

Isoprenol+ATP [symbol] isoprenyl monophosphate+ADP

The conversion of isoprenol into isoprenyl monophosphate according to this reaction can be achieved by enzymes which catalyze the transfer of a phospho group onto a molecule, such as kinases.

For example, enzymes which can be employed in this reaction are enzymes which are classified as E.C. 2.7.1, i.e. phosphotransferases with an alcohol group as acceptor, preferably enzymes which are classified as 2.7.1.50 (hydroxyethylthiazole kinase). Preferably, ATP is the donor of the phospho group in such a reaction. Thus, in one embodiment the enzymatic conversion of isoprenol into isoprenyl monophosphate can, e.g., be achieved by the use of a hydroxyethylthiazole kinase (EC 2.7.1.50). Hydroxyethylthiazole kinase is an enzyme which catalyzes the following reaction

ATP+4-methyl-5-(2-hydroxyethyl)thiazole⇄ADP+4-methyl-5-(2-phosphoethyl)thiazole

The occurrence of this enzyme has been described for several organisms, e.g. for E. coli, Bacillus subtilis, Rhizobium leguminosarum, Pyrococcus horikoshii OT3, Saccharomyces cerevisiae.

Hydroxyethylthiazole is a moiety of thiamine and shares with isoprenol some structural similarity. Thus, the inventors considered that a hydroxyethylthiazole kinase could also act on other substrates which contain this motif and found that, indeed, different tested hydroxyethylthiazole kinases were capable of using isoprenol as a substrate and converting it into isoprenyl monophosphate (see Examples 2 and 3).

In principle, any known hydroxyethylthiazole kinase can be employed in the method according to the invention. In one aspect of the present invention, a hydroxyethylthiazole kinase of bacterial origin is used, such as a hydroxyethylthiazole kinase from a bacterium belonging to the genus Escherichia, Bacillus or Rhizobium, preferably of E. coli, B. subtilis or of R. leguminosarum. Amino acid and nucleotide sequences for these enzymes are available. Examples are provided in SEQ ID NOs: 1 to 3.

### BRIEF DESCRIPTION OF THE DRAWINGS

FIG. 1 Chemical structure of isoprene.

FIG. 2 Metabolic reactions for isoprene production from isoprenol via isoprenyl or prenyl monophosphate.

FIG. 3 Metabolic reactions for isoprene production from isoprenol via isoprenyl sulfate or prenyl sulfate.

FIG. 4 Schematic representation of the ADP quantification assay. Assay is based on monitoring of NADH consumption through the decrease of absorbance at 340 nm.

FIG. 5A-B Electrospray MS spectrums of isoprenol phosphorylation reaction catalyzed by hydroethylthiazole kinase from R. leguminosarum (A), control assay without enzyme (B).

FIG. 6 Plot of the rate as a function of substrate concentration for the phosphotransferase reaction catalyzed by R. leguminosarum hydroxyethylthiazole kinase. Initial rates were computed from the kinetics over the 10 first minutes of the reaction.

FIG. 7 Isoprene production from isoprenyl monophosphate using terpene synthases.

FIG. 8A-B Mass spectrums of commercial isoprene (A) and isoprene produced from isopentenyl monophosphate in enzymatic reaction catalyzed by monoterpene synthase from Eucalyptus globulus (B). Characteristic ions of m/z 53, 67, 68, 69 representing isoprene were observed in both spectrums.

FIG. 9 Isoprene production from prenyl monophosphate using terpene synthases.

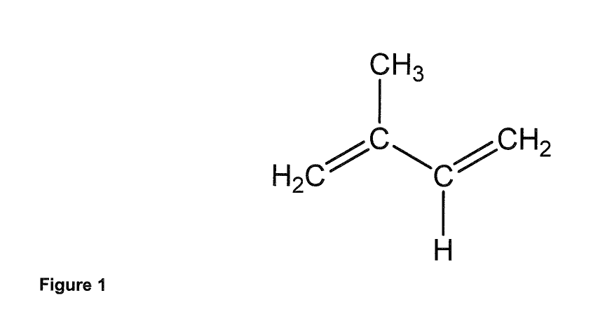

### DETAILED DESCRIPTION OF THE INVENTION

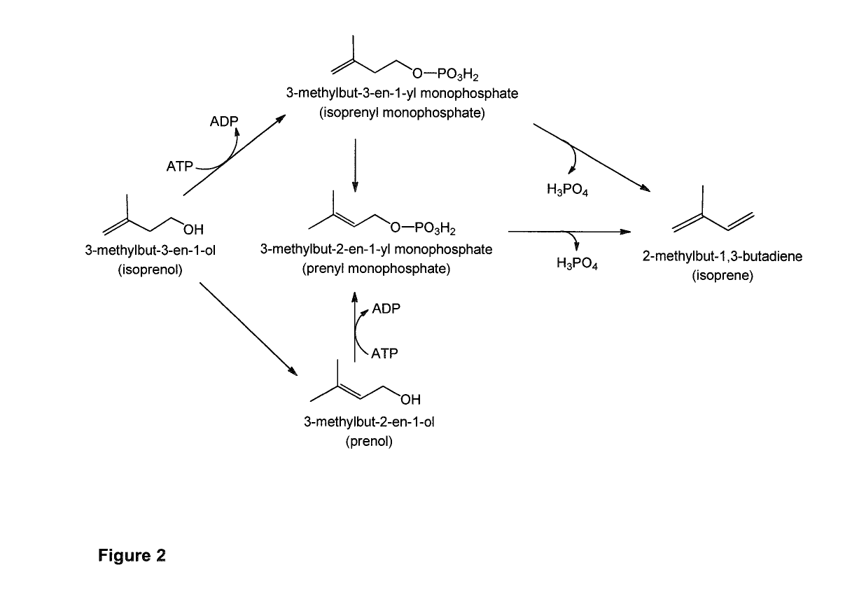

As described above, the obtained isoprenyl monophosphate is, according to the method of the present invention, further converted into isoprene. The enzymatic conversion of isoprenyl monophosphate into isoprene can be achieved by different routes which will be referred to in the following as Pathways A or B. Pathways A or B as described in the following are understood to comprise the enzymatic conversion of isoprenol into isoprenyl monophosphate as described herein above and, in addition, one of the pathways A or B as described in the following (see also FIG. 2) for converting isoprenyl monophosphate into isoprene.

Pathway A

According to Pathway A isoprenyl monophosphate is directly converted into isoprene by a dephosphorylation reaction according to the following scheme:

Isoprenyl monophosphate [symbol] isoprene+H₃PO₄

The direct enzymatic conversion of isoprenyl monophosphate into isoprene by this reaction can be achieved by the use of various enzymes, preferably by enzymes which are classified as terpene synthases.

The terpene synthases constitute an enzyme family which comprises enzymes catalyzing the formation of numerous natural products always composed of carbon and hydrogen (terpenes) and sometimes also of oxygen or other elements (terpenoids). Terpenoids are structurally diverse and widely distributed molecules corresponding to well over 30000 defined natural compounds that have been identified from all kingdoms of life. In plants, the members of the terpene synthase family are responsible for the synthesis of the various terpene molecules from two isomeric 5-carbon precursor “building blocks”, isoprenyl diphosphate and prenyl diphosphate, leading to 5-carbon isoprene, 10-carbon monoterpene, 15-carbon sesquiterpene and 20-carbon diterpenes” (Chen et al.; The Plant Journal 66 (2011), 212-229). The ability of terpene synthases to convert a prenyl diphosphate containing substrate to diverse products during different reaction cycles is one of the most unique traits of this enzyme class. The common key step for the biosynthesis of all terpenes is the reaction of terpene synthase on corresponding diphosphate esters. The general mechanism of this enzyme class induces the removal of the diphosphate group and the generation of an intermediate with carbocation as the first step. In the various terpene synthases, such intermediates further rearrange to generate the high number of terpene skeletons observed in nature. In particular, the resulting cationic intermediate undergoes a series of cyclizations, hydride shifts or other rearrangements until the reaction is terminated by proton loss or the addition of a nucleophile, in particular water for forming terpenoid alcohols (Degenhardt et al., Phytochemistry 70 (2009), 1621-1637).

The different terpene synthases share various structural features. These include a highly conserved C-terminal domain, which contains their catalytic site and an aspartate-rich DDXXD motif essential for the divalent metal ion (typically Mg2+ or Mn2+) assisted substrate binding in these enzymes (Green et al. Journal of biological chemistry, 284, 13, 8661-8669). In principle, any known enzyme which can be classified as belonging to the EC 4.2.3 enzyme superfamily can be employed.

In one embodiment of the present invention an isoprene synthase (EC 4.2.3.27) is used for the direct enzymatic conversion of isoprenyl monophosphate into isoprene. Isoprene synthase is an enzyme which catalyzes the following reaction:

Dimethylallyl diphosphate [symbol] isoprene+diphosphate

This enzyme occurs in a number of organisms, in particular in plants and some bacteria. The occurrence of this enzyme has, e.g., been described for Arabidopsis thaliana, a number of Populus species like P. alba (UniProt accession numbers Q50L36, A9Q7C9, D8UY75 and D8UY76), P. nigra (UniProt accession number A0PFK2), P. canescence (UniProt accession number Q9AR86; see also Köksal et al., J. Mol. Biol. 402 (2010), 363-373), P. tremuloides, P. trichocarpa (Seq ID NO: 4), in Quercus petraea, Quercus robur, Salix discolour, Pueraria montana (UniProt accession number Q6EJ97), Pueraria montana var. lobata (Seq ID NO: 5), Mucuna pruriens, Vitis vinifera, Embryophyta and Bacillus subtilis. In principle, any known isoprene synthase can be employed in the method according to the invention. In a preferred embodiment, the isoprene synthase employed in a method according to the present invention is an isoprene synthase from a plant of the genus Populus, more preferably from Populus trichocarpa or Populus alba. In another preferred embodiment the isoprene synthase employed in a method according to the present invention is an isoprene synthase from Pueraria montana, preferably from Pueraria montana var. lobata, or from Vitis vinifera. Preferred isoprene synthases to be used in the context of the present invention are the isoprene synthase of Populus alba (Sasaki et al.; FEBS Letters 579 (2005), 2514-2518) or the isoprene synthases from Populus trichocarpa and Populus tremuloides which show very high sequence homology to the isoprene synthase from Populus alba. Another preferred isoprene synthase is the isoprene synthase from Pueraria montana var. lobata (kudzu) (Sharkey et al.; Plant Physiol. 137 (2005), 700-712).

The activity of an isoprene synthase can be measured according to methods known in the art, e.g. as described in Silver and Fall (Plant Physiol (1991) 97, 1588-1591). In a typical assay, the enzyme is incubated with dimethylallyl diphosphate in the presence of the required co-factors, Mg²⁺ or Mn²⁺ and K⁺ in sealed vials. At appropriate time volatiles compound in the headspace are collected with a gas-tight syringe and analyzed for isoprene production by gas chromatography (GC).

Moreover, it is not only possible to use an isoprene synthase for converting isoprenol into isoprene according to the above shown scheme, but it is also possible to use other enzymes from the family of monoterpene synthases. Monoterpene synthases comprise a number of families to which specific EC numbers are allocated. However, they also include also a number of enzymes which are simply referred to as monoterpene synthases and which are not classified into a specific EC number. To the latter group belong, e.g., the monoterpene synthases of Eucalyptus globulus (UniProt accession number Q0PCI4) and of Melaleuca alternifolia described in Shelton et al. (Plant Physiol. Biochem. 42 (2004), 875-882). In particularly preferred embodiments of the present invention use is made of a monoterpene synthase of Eucalyptus globulus (SEQ ID NO: 6) or of Melaleuca alternifolia (Seq ID NO: 7).

In other preferred embodiments of the method according to the invention the conversion of isoprenol into isoprene according to the above shown scheme is achieved by a terpene synthase belonging to one of the following families: alpha-farnesene synthases (EC 4.2.3.46), beta-farnesene synthases (EC 4.2.3.47), myrcene/(E)-beta-ocimene synthases (EC 4.2.3.15) and pinene synthase (EC 4.2.3.14).

Farnesene synthases are generally classified into two different groups, i.e. alpha-farnesene synthases (EC 4.2.3.46) and beta farnesene synthases (EC 4.2.3.47). Alpha-farnesene synthases (EC 4.2.3.46) naturally catalyze the following reaction:

(2E,6E)-farnesyl diphosphate [symbol] (3E,6E)-alpha-famesene+diphosphate

This enzyme occurs in a number of organisms, in particular in plants, for example in Malus×domestica (UniProt accession numbers Q84LB2, B2ZZ11, Q6Q2J2, Q6QWJ1 and Q32WI2), Populus trichocarpa, Arabidopsis thaliana (UniProt accession numbers A4FVP2 and P0CJ43), Cucumis melo (UniProt accession number B2KSJ5) and Actinidia deliciosa (UniProt accession number C7SHN9). In principle, any known alpha-farnesene synthase can be employed in the method according to the invention. In a preferred embodiment, the alpha-farnesene synthase employed in a method according to the present invention is an alpha-farnesene synthase from Malus×domestica (e.g. Seq ID NO:8), UniProt accession numbers Q84LB2, B2ZZ11, Q6Q2J2, Q6QWJ1 and Q32WI2; see also Green et al.; Photochemistry 68 (2007), 176-188).

Beta-farnesene synthases (EC 4.2.3.47) naturally catalyze the following reaction:

(2E,6E)-farnesyl diphosphate [symbol] (E)-beta-farnesene+diphosphate

This enzyme occurs in a number of organisms, in particular in plants and in bacteria, for example in Artemisia annua (UniProt accession number Q4VM12), Citrus junos (UniProt accession number Q94JS8), Oryza sativa (UniProt accession number Q0J7R9), Pinus sylvestris (UniProt accession number D7PCH9), Zea diploperennis (UniProt accession number C7E5V9), Zea mays (UniProt accession numbers Q2NM15, C7E5V8 and C7E5V7), Zea perennis (UniProt accession number C7E5W0) and Streptococcus coelicolor (Zhao et al., J. Biol. Chem. 284 (2009), 36711-36719). In principle, any known beta-farnesene synthase can be employed in the method according to the invention. In a preferred embodiment, the beta-farnesene synthase employed in a method according to the present invention is a beta-farnesene synthase from Mentha piperita (Crock et al.; Proc. Natl. Acad. Sci. USA 94 (1997), 12833-12838).

Methods for the determination of farnesene synthase activity are known in the art and are described, for example, in Green et al. (Phytochemistry 68 (2007), 176-188). In a typical assay farnesene synthase is added to an assay buffer containing 50 mM BisTrisPropane (BTP) (pH 7.5), 10% (v/v) glycerol, 5 mM DTT. Tritiated farnesyl diphosphate and metal ions are added. Assays containing the protein are overlaid with 0.5 ml pentane and incubated for 1 h at 30° C. with gentle shaking. Following addition of 20 mM EDTA (final concentration) to stop enzymatic activity an aliquot of the pentane is removed for scintillation analysis. The olefin products are also analyzed by GC-MS.

Myrcene/(E)-beta-ocimene synthases (EC 4.2.3.15) are enzymes which naturally catalyze the following reaction:

Geranyl diphosphate [symbol] (E)-beta-ocimene+diphosphate

or

Geranyl diphosphate [symbol] myrcene+diphosphate

These enzymes occur in a number of organisms, in particular in plants and animals, for example in Lotus japanicus (Arimura et al.; Plant Physiol. 135 (2004), 1976-1983), Phaseolus lunatus (UniProt accession number B1P189), Abies grandis, Arabidopsis thaliana (UniProt accession number Q9ZUH4), Actinidia chinensis, Vitis vinifera (E5GAG5), Perilla fructescens, Ochtodes secundiramea and in Ips pini (UniProt accession number Q58GE8). In principle, any known myrcene/ocimene synthase can be employed in the method according to the invention. In a preferred embodiment, the myrcene/ocimene synthase employed in a method according to the present invention is an (E)-beta-ocimene synthase from Vitis vinifera (Seq ID NO: 9).

The activity of an ocimene/myrcene synthase can be measured as described, for example, in Arimura et al. (Plant Physiology 135 (2004), 1976-1983). In a typical assay for determining the activity, the enzyme is placed in screwcapped glass test tube containing divalent metal ions, e.g. Mg²⁺ and/or Mn²⁺, and substrate, i.e. geranyl diphosphate. The aqueous layer is overlaid with pentane to trap volatile compounds. After incubation, the assay mixture is extracted with pentane a second time, both pentane fractions are pooled, concentrated and analyzed by gas chromatography to quantify ocimene/myrcene production.

Pinene synthase (EC 4.2.3.14) is an enzyme which naturally catalyzes the following reaction:

Geranyl diphosphate [symbol] alpha-pinene+diphosphate

This enzyme occurs in a number of organisms, in particular in plants, for example in Abies grandis (UniProt accession number 024475), Artemisia annua, Chamaecyparis formosensis (UniProt accession number C3RSF5), Salvia officinalis and Picea sitchensis (UniProt accession number Q6XDB5).

For the enzyme from Abies grandis a particular reaction was also observed (Schwab et al., Arch. Biochem. Biophys. 392 (2001), 123-136), namely the following:

6,7-dihydrogeranyl diphosphate [symbol] 6,7-dihydromyrcene+diphosphate

In principle, any known pinene synthase can be employed in the method according to the invention. In a preferred embodiment, the pinene synthase employed in a method according to the present invention is a pinene synthase from Abies grandis (UniProt accession number 024475; Schwab et al., Arch. Biochem. Biophys. 392 (2001), 123-136).

Methods for the determination of pinene synthase activity are known in the art and are described, for example, in Schwab et al. (Archives of Biochemistry and Biophysics 392 (2001), 123-136). In a typical assay, the assay mixture for pinene synthase consists of 2 ml assay buffer (50 mM Tris/HCl, pH 7.5, 500 mM KCl, 1 mM MnCl2, 5 mM dithiothreitol, 0.05% NaHSO3, and 10% glycerol) containing 1 mg of the purified protein. The reaction is initiated in a Teflon-sealed screw-capped vial by the addition of 300 mM substrate. Following incubation at 25° C. for variable periods (0.5-24 h), the mixture is extracted with 1 ml of diethyl ether. The biphasic mixture is vigorously mixed and then centrifuged to separate the phases. The organic extract is dried (MgSO4) and subjected to GC-MS and MDGC analysis.

Pathway B

According to Pathway B isoprenyl monophosphate is first converted enzymatically into prenyl monophosphate by an isomerisation reaction and prenyl monophosphate is then in a second enzymatic step converted into isoprene by a dephosphorylation reaction according to the following scheme:

Isoprenyl monophosphate [symbol] prenyl monophosphate

Prenyl monophosphate [symbol] isoprene+H₃PO₄

The enzymatic conversion of isoprenyl monophosphate to prenyl monophosphate can, e.g., be achieved by the use of an enzyme which is classified as an isopentenyl-diphosphate DELTA isomerase (EC 5.3.3.2). Isopentenyl-diphosphate DELTA isomerise catalyzes the following reaction:

Isopentenyl diphosphate⇄dimethylallyl diphosphate

The occurrence of this enzyme has been described for a large number of organisms, e.g. for E. coli, Staphylococcus aureus, Sulfolobus shibatae, Bacillus subtilis, Thermococcus kodakarensis, Solanum lycopersicum, Arabidopsis thaliana, Bombyx mori, Camptotheca acuminata, Capsicum annuum, Catharanthus roseus, Cinchona robusta, Citrus sp., Claviceps purpurea, Curcubita sp., Gallus gallus and Homo sapiens, to name just some. In a preferred embodiment, the enzyme originating from E. coli or an enzyme derived therefrom and which still shows the activity as the enzyme from E. coli is employed in the methods according to the present invention.

The conversion of prenyl monophosphate into isoprene according to the above given scheme can, e.g., be achieved by the use of terpene synthases, in particular by the use of an isoprene synthase (EC 4.2.3.27) or another terpene synthase. Such enzymes have already been described above and the same as described above also applies here.

In another aspect, the present invention relates to a method for the production of isoprene in which isoprenol is first enzymatically converted into prenol by an isomerisation reaction, prenol is then converted in a second enzymatic step into prenyl monophosphate by a phosphorylation reaction and prenyl monophosphate is then further enzymatically converted into isoprene (see FIG. 2). This conversion of isoprenol into isoprene will be referred to in the following as Pathway C.

Pathway C

According to Pathway C prenyl monophosphate is directly converted into isoprene by a dephosphorylation reaction. Thus, according to Pathway C the overall reaction scheme is as follows:

Isoprenol [symbol] prenol

Prenol+ATP [symbol] prenyl monophosphate+ADP

Prenyl monophosphate [symbol] isoprene+H₃PO₄

The enzymatic conversion of isoprenol to prenol can, e.g., be achieved by the use of an enzyme which is classified as an isopentenyl-diphosphate DELTA isomerase (EC 5.3.3.2). This enzyme has already been described in connection with Pathway B and the same applies here.

The conversion of prenol into prenyl monophosphate according to the above shown reaction can be achieved by enzymes which catalyze the transfer of a phospho group onto a molecule, such as kinases.

For example, enzymes which can be employed in this reaction are enzymes which are classified as E.C. 2.7.1, i.e. phosphotransferases with an alcohol group as acceptor, preferably enzymes which are classified as 2.7.1.50 (hydroxyethylthiazole kinase). Preferably, ATP is the donor of the phospho group in such a reaction. The corresponding enzymes have already been described herein above and the same applies here. The inventors could show that hydroxyethylthiazole kinase is indeed capable of converting prenol into prenyl monophosphate (see Example 4).

The conversion of prenyl monophosphate into isoprene according to the above given scheme can, e.g., be achieved by the use of terpene synthases, in particular by the use of an isoprene synthase (EC 4.2.3.27) or another terpene synthase. Such enzymes have already been described above and the same as described above also applies here.

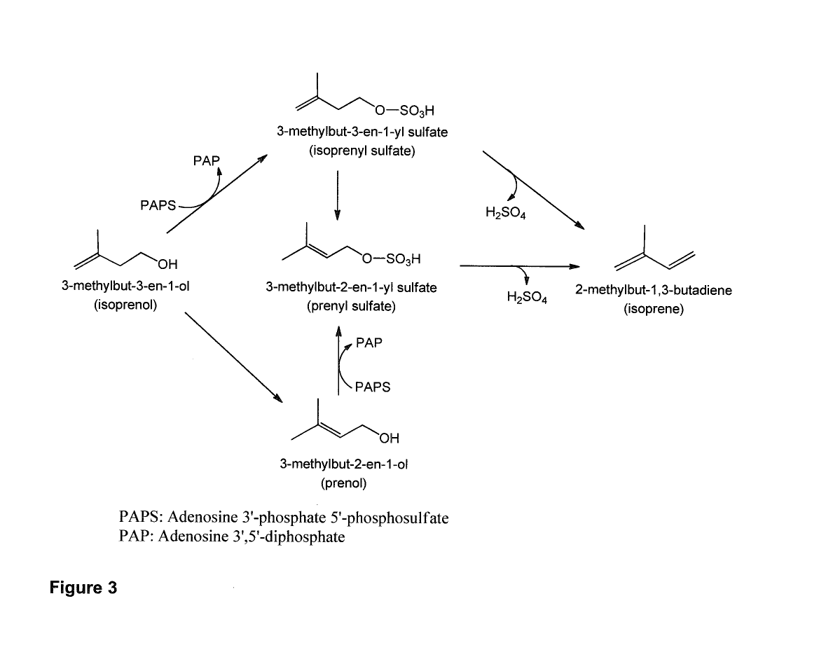

In another aspect, the present invention relates to a method for the production of isoprene in which isoprenol is first enzymatically converted into isoprenyl sulfate and in which isoprenyl sulfate is then further converted into isoprene according to the following general scheme (see also FIG. 3):

Isoprenol [symbol] isoprenyl sulfate [symbol] isoprene

According to the present invention the conversion of isoprenol into isoprenyl sulfate occurs preferably according to the following reaction:

Isoprenol+PAPS [symbol] isoprenyl sulfate+PAP

wherein PAPS stands for adenosine 3′-phosphate 5′-phosphosulfate and PAP stands for adenosine 3′,5′-diphosphate.

The conversion of isoprenol into isoprenyl sulfate according to this reaction can be achieved by enzymes which catalyze the transfer of a sulfate group onto a molecule, such as sulfotransferases.

For example, enzymes which can be employed in this reaction are enzymes which are classified as E.C. 2.8.2, i.e. transferase enzymes that catalyze the transfer of a sulfate group from a donor molecule to an acceptor alcohol or amine. Preferably, PAPS is the donor of the sulfate group in such a reaction. In principle, any sulfotransferase can be used. In a preferred embodiment the sulfotransferase is an alcohol sulfotransferase (EC 2.8.2.2), a steroid sulfotransferase (EC 2.8.2.15), a scymnol sulfotransferase (EC 2.8.2.32), a flavonol 3-sulfotransferase (EC 2.8.2.25) or a retinol sulfotransferase/dehydratase.

Thus, in one preferred embodiment the enzymatic conversion of isoprenol into isoprenyl sulfate can, e.g., be achieved by the use of an alcohol sulfotransferase (EC 2.8.2.2). Alcohol sulfotransferases are enzymes which catalyze the following reaction:

3′-phosphoadenylyl sulfate (PAPS)+an alcohol/ [symbol] adenosine3′,5′-bisphosphate (PAP)+an alkyl sulfate

The occurrence of these enzymes has been described for a number of organisms, e.g. for E. coli, Oryctolagus cuniculus, Petromyzon marinus, Rana catesbeiana, Rattus norvegicus, Mus musculus, Cavia porcellus, Mesocricetus auratus, Sus scrofa, Drosophila melanogaster and Homo sapiens. In principle, any known alcohol sulfotransferase can be employed in the method according to the invention. In one aspect of the present invention, a alcohol sulfotransferase of mammalian origin is used, such as a alcohol sulfotransferase from an organism belonging to the genus Rattus, preferably of the species Rattus norvegicus (Lyon and Jakoby; Arch. Biochem. Biophys. 202 (1980), 474-481).

In another preferred embodiment the enzymatic conversion of isoprenol into isoprenyl sulfate can, e.g., be achieved by the use of a steroid sulfotransferase (EC 2.8.2.15). Steroid sulfotransferases are enzymes which catalyze the following reaction:

3′-phosphoadenylyl sulfate (PAPS)+a phenolic steroid [symbol] adenosine3′,5′-bisphosphate (PAP)+a steroid O-sulfate

The occurrence of these enzymes has been described for a number of organisms, e.g. for Rattus norvegicus, Mus musculus, Cavia porcellus, Sus scrofa, Danio rerio, Bos Taurus, Brassica napus and Homo sapiens. In principle, any known steroid sulfotransferase can be employed in the method according to the invention.

In another preferred embodiment the enzymatic conversion of isoprenol into isoprenyl sulfate can, e.g., be achieved by the use of a scymnol sulfotransferase (EC 2.8.2.32). Scymnol sulfotransferases are enzymes which catalyze the following reaction:

3′-phosphoadenylyl sulfate (PAPS)+5-beta scymnol [symbol] adenosine3′,5′-bisphosphate (PAP)+5-beta scymnol sulfate

The occurrence of these enzymes has been described for some organisms, e.g. for Heterodontus portusjacksoni, Trygonorrhina fasciata and Trygonoptera sp. In principle, any known scymnol sulfotransferase can be employed in the method according to the invention.

In another preferred embodiment the enzymatic conversion of isoprenol into isoprenyl sulfate can, e.g., be achieved by the use of a flavonol 3-sulfotransferase (EC 2.8.2.25). Flavonol sulfotransferases are enzymes which catalyze the following reaction:

3′-phosphoadenylyl sulfate (PAPS)+quercetin/ [symbol] adenosine3′,5′-bisphosphate (PAP)+quercetin3-sulfate

Apart from quercetin, these enzymes also accept other flavonol aglycones as substrate.

The occurrence of these enzymes has been described for some organisms, e.g. for Flaveria chlorifolia and Flavera bidentis. In principle, any known flavonol sulfotransferase can be employed in the method according to the invention.

In another preferred embodiment the enzymatic conversion of isoprenol into isoprenyl sulfate can, e.g., be achieved by the use of an enzyme which is classified as a retinol sulfotransferase/dehydratase. This enzyme is, e.g., described in Pakhomova et al. (Protein Science 14 (2005), 176-182) and in Vakiani et al. (J. Biol. Chem. 273 (1998), 35381-35387). This enzyme catalyzes the conversion of retinol to the retro-retinoid anhydro-retinol according to the following reaction:

Retinol+PAPS=>retinyl sulfate+PAP=>anhydroretinol

It belongs to the sulfotransferase superfamily but shows some unique features that distinguish it from other members of this superfamily. It has only a very low sequence homology to the most homologous sulfotransferase rat aryl sulfotransferase (30%) and it is significantly larger (41 kDa) than mammalian sulfotransferases (30-36 kDa). It is a typical cytosolic sulfotransferase and sulfonates a wide variety of different hydroxycompounds, such as p-nitrophenol, phenol, vanillin and serotonin. The feature that most distinguishes the enzyme from other sulfotransferases is that the end product of the enzymatic reaction, anhydroretinol, is not sulfonated. Retinyl sulfate appears to be a transient intermediate in the transformation of retinol to anhydroretinol.

In a preferred embodiment the retinol sulfotransferase/dehydratase employed in a method according to the invention is a retinol sulfotransferase/dehydratase from Spodoptera frugiperda (Uniprot accession number Q26490) or from Danaus plexippus (Uniprot accession number G6DMT5).

As described above, the obtained isoprenyl sulfate is, according to the method of the present invention, further converted into isoprene. This second conversion can either be achieved by a thermal conversion or by an enzymatic reaction as will be explained in more detail in the following.

In a first aspect of the method according to the invention, the conversion of isoprenyl sulfate into isoprene is achieved by a thermal conversion. “Thermal conversion” in this context means that the isoprenyl sulfate is incubated at elevated temperatures. It is expected that incubation of isoprenyl sulfate at elevated temperatures leads to a significant conversion into isoprene. The term “elevated temperature” means a temperature which is higher than room temperature, preferably 30° C. or higher and more preferably about 37° C. or higher. This opens up the possibility to employ in a method according to the invention mesophilic (micro)organisms which can be cultured at these temperatures, such as e.g. E. coli. In other embodiments higher temperatures may be employed to achieve the conversion of the isoprenyl sulfate into isoprene. Accordingly, in other preferred embodiments the term “elevated temperature” means a temperature which is 40° C. or higher, more preferably 45° C. or higher, even more preferably 50° C. or higher, 55° C. or higher, 60° C. or higher and particularly preferred 65° C. or higher.

In another aspect of the method according to the invention, the conversion of isoprenyl sulfate into isoprene is achieved by an enzymatic reaction, in particular by a desulfurylation. The enzymatic conversion of isoprenyl sulfate into isoprene can be achieved by different routes which will be referred to in the following as Pathways D or E. Pathways D or E as described in the following are understood to comprise the enzymatic conversion of isoprenol into isoprenyl sulfate as described herein above and, in addition, one of the pathways D or E as described in the following (see also FIG. 3) for converting isoprenyl sulfate into isoprene.

Pathway D

According to Pathway D isoprenyl sulfate is directly converted into isoprene by a desulfurylation reaction according to the following scheme:

Isoprenyl sulfate [symbol] isoprene+H₂SO₄

The direct enzymatic conversion of isoprenyl sulfate into isoprene by this reaction can be achieved by the use of various enzymes, preferably by enzymes which are classified as terpene synthases or by a retinol sulfotransferase/dehydratase. These enzymes have been described in detail herein above in connection with Pathways A and C, respectively, and the same as said above also applies here.

Pathway E

According to Pathway E isoprenyl sulfate is first converted enzymatically into prenyl sulfate by an isomerisation reaction and prenyl sulfate is then in a second enzymatic step converted into isoprene by a desulfurylation reaction according to the following scheme:

Isoprenyl sulfate [symbol] prenyl sulfate

Prenyl sulfate [symbol] isoprene+H₂SO₄

The enzymatic conversion of isoprenyl sulfate to prenyl sulfate can, e.g., be achieved by the use of an enzyme which is classified as an isopentenyl-diphosphate DELTA isomerase (EC 5.3.3.2). This enzyme has been described above in connection with Pathway B and the same that has been said above also applies here.

The conversion of prenyl sulfate into isoprene according to the above given scheme can either be achieved by a thermal conversion or by an enzymatic reaction as will be explained in more detail in the following.

In a first aspect of a method according to the invention, the conversion of prenyl sulfate into isoprene is achieved by a thermal conversion. “Thermal conversion” in this context means that the prenyl sulfate is incubated at elevated temperatures. It is expected that incubation of prenyl sulfate at elevated temperatures leads to a significant conversion into isoprene. The term “elevated temperature” means a temperature which is higher than room temperature, preferably 30° C. or higher and more preferably about 37° C. or higher. This opens up the possibility to employ in a method according to the invention mesophilic (micro)organisms which can be cultured at these temperatures, such as e.g. E. coli. In other embodiments higher temperatures may be employed to achieve the conversion of the prenyl sulfate into isoprene. Accordingly, in other preferred embodiments the term “elevated temperature” means a temperature which is 40° C. or higher, more preferably 45° C. or higher, even more preferably 50° C. or higher, 55° C. or higher, 60° C. or higher and particularly preferred 65° C. or higher.

In another aspect the conversion of prenyl sulfate into isoprene according to the above given scheme is achieved by the use of terpene synthases, in particular by the use of an isoprene synthase (EC 4.2.3.27) or another terpene synthase, or by the use of a retinol sulfotransferase/dehydratase. Such enzymes have already been described above and the same as described above also applies here.

In another aspect, the present invention relates to a method for the production of isoprene in which isoprenol is first enzymatically converted into prenol by an isomerisation reaction, prenol is then converted in a second enzymatic step into prenyl sulfate by a sulfurylation reaction and prenyl sulfate is then further enzymatically converted into isoprene (see FIG. 3). This conversion of isoprenol into isoprene will be referred to in the following as Pathway F.

Pathway F

According to Pathway F prenyl sulfate is directly converted into isoprene by a desulfurylation reaction. Thus, according to Pathway F the overall reaction scheme is as follows:

Isoprenol [symbol] prenol

Prenol+PAPS [symbol] prenyl sulfate+PAP

Prenyl sulfate [symbol] isoprene+H₂SO₄

The enzymatic conversion of isoprenol to prenol can, e.g., be achieved by the use of an enzyme which is classified as an isopentenyl-diphosphate DELTA isomerase (EC 5.3.3.2). This enzyme has already been described in connection with Pathway B and the same applies here.

The conversion of prenol into prenyl sulfate according to the above shown reaction can be achieved by enzymes which catalyze the transfer of a sulfate group onto a molecule, such as sulfotransferases.

For example, enzymes which can be employed in this reaction are enzymes which are classified as E.C. 2.8.2, i.e. transferase enzymes that catalyze the transfer of a sulfate group from a donor molecule to an acceptor alcohol or amine. Preferably, PAPS is the donor of the sulfate group in such a reaction. In principle, any sulfotransferase can be used. In a preferred embodiment the sulfotransferase is an alcohol sulfotransferase (EC 2.8.2.2), a steroid sulfotransferase (EC 2.8.2.15), a scymnol sulfotransferase (EC 2.8.2.32), a flavonol 3-sulfotransferase (EC 2.8.2.25) or a retinol sulfotransferase/dehydratase. These enzymes have been described in detail above and the same applies here.

The conversion of prenyl sulfate into isoprene according to the above given scheme can, e.g., be achieved as described above, e.g. by thermal conversion or by the use of enzymes, preferably by the use of terpene synthases, in particular by the use of an isoprene synthase (EC 4.2.3.27) or another terpene synthase, or by the use of a retinol sulfotransferase/dehydratase. Such enzymes have already been described above and the same as described above also applies here.

The enzymes employed in the different reactions according to the methods according to the invention as described above, can be a naturally occurring enzymes or they can be enzymes which are derived from a naturally occurring enzyme e.g. by the introduction of mutations or other alterations which, e.g., alter or improve the enzymatic activity, the stability, etc.

When the present invention refers to a certain enzyme to be used for a conversion of a substrate in a reaction in one of the Pathways of a method according to the invention, such reference to an enzyme also covers enzymes which are derived from such an enzyme, which are capable of catalyzing the reaction as indicated for a certain Pathway of the present invention but which only have a low affinity to their natural substrate or do no longer accept their natural substrate.

Such a modification of the preferred substrate of an enzyme to be employed in a method according to the present invention allows to improve the conversion of the respective substrate of a reaction of a method according to the present invention and to reduce the production of unwanted by-product(s) due to the action of the enzyme on their natural substrate(s). Methods for modifying and/or improving the desired enzymatic activities of proteins are well-known to the person skilled in the art and include, e.g., random mutagenesis or site-directed mutagenesis and subsequent selection of enzymes having the desired properties or approaches of the so-called “directed evolution”.

For example, for genetic engineering in prokaryotic cells, a nucleic acid molecule encoding an enzyme as employed in a method according to the present invention can be introduced into plasmids which permit mutagenesis or sequence modification by recombination of DNA sequences. Standard methods (see Sambrook and Russell (2001), Molecular Cloning: A Laboratory Manual, CSH Press, Cold Spring Harbor, N.Y., USA) allow base exchanges to be performed or natural or synthetic sequences to be added. DNA fragments can be connected to each other by applying adapters and linkers to the fragments. Moreover, engineering measures which provide suitable restriction sites or remove surplus DNA or restriction sites can be used. In those cases, in which insertions, deletions or substitutions are possible, in vitro mutagenesis, “primer repair”, restriction or ligation can be used. In general, a sequence analysis, restriction analysis and other methods of biochemistry and molecular biology are carried out as analysis methods. The resulting enzyme variants are then tested for their enzymatic activity and in particular for their capacity to convert a substrate as indicated in the respective reaction of a method according to the invention as a substrate rather than their natural substrate(s) as described above in connection with the description of the different enzymes which can be used in the context of the methods according to the present invention.

Assays for measuring the capacity of an enzyme to catalyze a reaction as indicated in connection with a Pathway of a method according to the invention are described in the Examples.

The modified version of the enzyme having a low affinity to its natural substrate or no longer accepting its natural substrate may be derived from a naturally occurring enzyme or from an already modified, optimized or synthetically produced enzyme.

The enzyme employed in the process according to the present invention can be a natural version of the protein or a synthetic protein as well as a protein which has been chemically synthesized or produced in a biological system or by recombinant processes. The enzyme may also be chemically modified, for example in order to improve its/their stability, resistance, e.g. to temperature, for facilitating its purification or its immobilization on a support. The enzyme may be used in isolated form, purified form, in immobilized form, as a crude or partially purified extract obtained from cells synthesizing the enzyme, as chemically synthesized enzyme, as recombinantly produced enzyme, in the form of microorganisms producing them etc.

The methods according to the present invention may be carried out in vitro or in vivo. An in vitro reaction is understood to be a reaction in which no cells are employed, i.e. an acellular reaction. Thus, in vitro preferably means in a cell-free system. The term “in vitro” in one embodiment means in the presence of isolated enzymes (or enzyme systems optionally comprising possibly required cofactors). In one embodiment, the enzymes employed in the method are used in purified form.

For carrying out the process in vitro the substrates for the reaction and the enzymes are incubated under conditions (buffer, temperature, cosubstrates, cofactors etc.) allowing the enzymes to be active and the enzymatic conversion to occur. The reaction is allowed to proceed for a time sufficient to produce isoprene. The production of isoprene can be measured by methods known in the art, such as gas chromatography possibly linked to mass spectrometry detection.

The enzymes may be in any suitable form allowing the enzymatic reaction to take place. They may be purified or partially purified or in the form of crude cellular extracts or partially purified extracts. It is also possible that the enzymes are immobilized on a suitable carrier.

The in vitro method according to the invention may be carried out in a one-pot-reaction, i.e. the substrate is combined in one reaction mixture with the above described enzymes necessary for the conversion into isoprene and the reaction is allowed to proceed for a time sufficient to produce isoprene. Alternatively, the method may also be carried out by effecting one or more enzymatic steps in a consecutive manner, i.e. by first mixing the substrate with one or more enzymes and allowing the reaction to proceed to an intermediate and then adding one or more further enzymes to convert the intermediate further either into an intermediate or into isoprene.

The recovery of isoprene may involve one step or multiples steps. For example, isoprene can be recovered using standard techniques such as adsorption/desorption, gas stripping, fractionation. Separation of isoprene from CO₂ can be achieved by the condensation of CO₂ at low temperature. CO₂ can also be removed by polar solvents, e.g. ethanolamine.

In another embodiment the method according to the invention is carried out in culture, in the presence of an organism, preferably a microorganism, producing at least the enzymes described above which are necessary to produce isoprene according to a method of the invention involving any one of Pathways A to F as described herein above. Such organisms or microorganisms are also an object of the present invention.

If a (micro)organism is used which naturally expresses one of the required enzyme activities, it is possible to modify such a (micro)organism so that this activity is overexpressed in the (micro)organism. This can, e.g., be achieved by effecting mutations in the promoter region of the corresponding gene so as to lead to a promoter which ensures a higher expression of the gene. Alternatively, it is also possible to mutate the gene as such so as to lead to an enzyme showing a higher activity.

By using (micro)organisms which express the enzymes which are necessary according to any one of the Pathways A to F as described herein above, it is possible to carry out the method according to the invention directly in the culture medium, without the need to separate or purify the enzymes.

In one embodiment, a (micro)organism is used having the natural or artificial property of endogenously producing isoprenol, and also expressing or overexpressing the enzymes as described in connection with Pathways A to F, above, so as to produce isoprene from a carbon source present in solution.

In one embodiment the (micro)organism according to the present invention or employed in the method according to the invention is an organism, preferably a microorganism, which has been genetically modified to contain one or more foreign nucleic acid molecules encoding one or more of the enzymes as described above in connection with Pathways A to F. The term “foreign” in this context means that the nucleic acid molecule does not naturally occur in said organism/microorganism. This means that it does not occur in the same structure or at the same location in the organism/microorganism. In one preferred embodiment, the foreign nucleic acid molecule is a recombinant molecule comprising a promoter and a coding sequence encoding the respective enzyme in which the promoter driving expression of the coding sequence is heterologous with respect to the coding sequence. Heterologous in this context means that the promoter is not the promoter naturally driving the expression of said coding sequence but is a promoter naturally driving expression of a different coding sequence, i.e., it is derived from another gene, or is a synthetic promoter or a chimeric promoter. Preferably, the promoter is a promoter heterologous to the organism/microorganism, i.e. a promoter which does naturally not occur in the respective organism/microorganism. Even more preferably, the promoter is an inducible promoter. Promoters for driving expression in different types of organisms, in particular in microorganisms, are well known to the person skilled in the art.

In a further embodiment the nucleic acid molecule is foreign to the organism/microorganism in that the encoded enzyme is not endogenous to the organism/microorganism, i.e. is naturally not expressed by the organism/microorganism when it is not genetically modified. In other words, the encoded enzyme is heterologous with respect to the organism/microorganism. The foreign nucleic acid molecule may be present in the organism/microorganism in extrachromosomal form, e.g. as a plasmid, or stably integrated in the chromosome. A stable integration is preferred. Thus, the genetic modification can consist, e.g. in integrating the corresponding gene(s) encoding the enzyme(s) into the chromosome, or in expressing the enzyme(s) from a plasmid containing a promoter upstream of the enzyme-coding sequence, the promoter and coding sequence preferably originating from different organisms, or any other method known to one of skill in the art.

In a preferred embodiment the (micro)organism of the present invention is also genetically modified so as to be able to produce isoprenol. Ways of genetically modifying (micro)organisms so as to be able to produce isoprenol are, e.g., described in WO 2011/076261. Thus, in a preferred embodiment, a (micro)organism of the present invention or employed in a method according to the present invention is capable of converting mevalonate into isoprenol by a decarboxylation reaction. Preferably such a (micro)organism expresses an enzyme which is classified as a diphosphomevalonate decarboxylase or is an enzyme which is derived from such an enzyme and which has the capacity to decarboxylate mevalonate so as to produce isoprenol. Diphosphomevalonate decarboxylase is classified with the EC number EC 4.1.1.33.

The organisms used in the invention can be prokaryotes or eukaryotes, preferably, they are microorganisms such as bacteria, yeasts, fungi or molds, or plant cells or animal cells. In a particular embodiment, the microorganisms are bacteria, preferably of the genus Escherichia or Bacillus and even more preferably of the species Escherichia coli or Bacillus subtilis.

In another embodiment, the microorganisms are recombinant bacteria of the genus Escherichia or Bacillus, preferably of the species Escherichia coli or Bacillus subtilis, having been modified so as to endogenously produce isoprenol and to convert it into isoprene.

It is also possible to employ an extremophilic bacterium such as Thermus thermophilus, or anaerobic bacteria from the family Clostridiae.

In one embodiment the microorganism is a fungus, more preferably a fungus of the genus Saccharomyces, Schizosaccharomyces, Aspergillus, Trichoderma, Pichia or Kluyveromyces and even more preferably of the species Saccharomyces cerevisiae, Schizosaccharomyces pombe, Aspergillus niger, Trichoderma reesei, Pichia pastoris or of the species Kluyveromyces lactis. In a particularly preferred embodiment the microorganism is a recombinant yeast capable of producing isoprenol and converting it into isoprene due to the expression of the enzymes described in connection with any one of Pathways A to F, above.

In another embodiment, the method according to the invention makes use of a photosynthetic microorganism expressing at least the enzymes as described in connection with any one of Pathways A to F, above. Preferably, the microorganism is a photosynthetic bacterium, or a microalgae. In a further embodiment the microorganism is an algae, more preferably an algae belonging to the diatomeae.

Even more preferably such a microorganism has the natural or artificial property of endogenously producing isoprenol. In this case the microorganism would be capable of producing isoprenol directly from CO₂ present in solution.

In another embodiment, the microorganism is a microorganism which belongs to the group of acetogenic bacteria which are capable of converting CO (or CO₂+H₂) to produce acetyl-CoA via the so-called Wood-Ljungdahl pathway (Köpke et al.; PNAS 10 (2010), 13087-13092). A fermentation process using such microorganisms is known as syngas fermentation. Strictly mesophilic anaerobes such as C. ljungdahlii, C. aceticum, Acetobacterium woodii, C. autoethanogenum, and C. carboxydeviron, are frequently being used in syngas fermentation (Munasingheet et al.; Bioresource Technology 101 (2010), 5013-5022).

It is also conceivable to use in the method according to the invention a combination of (micro)organisms wherein different (micro)organisms express different enzymes as described above. In a further embodiment at least one of the microorganisms is capable of producing isoprenol or, in an alternative embodiment, a further microorganism is used in the method which is capable of producing isoprenol.

In another embodiment the method according to the invention makes use of a multicellular organism expressing at least the enzymes as described in connection with any one of Pathways A to F, above. Examples for such organisms are plants or animals.

In a particular embodiment, the method according to the invention involves culturing microorganisms in standard culture conditions (30-37° C. at 1 atm, in a fermenter allowing aerobic growth of the bacteria) or non-standard conditions (higher temperature to correspond to the culture conditions of thermophilic organisms, for example).

In a further embodiment the method of the invention is carried out under conditions under which the produced isoprene is in a gaseous state. In such a case, it is furthermore preferred that the method is carried out under microaerophilic conditions. This means that the quantity of injected air is limiting so as to minimize residual oxygen concentrations in the gaseous effluents containing isoprene.

In another embodiment the method according to the invention furthermore comprises the step of collecting the gaseous isoprene degassing out of the reaction. Thus in a preferred embodiment, the method is carried out in the presence of a system for collecting isoprene under gaseous form during the reaction.

As a matter of fact, isoprene adopts the gaseous state at temperatures of more than about 34° C. and atmospheric pressure. The method according to the invention when carried out under conditions which allow isoprene to be in the gaseous state, therefore does not require extraction of isoprene from the liquid culture medium, a step which is always very costly when performed at industrial scale. The evacuation and storage of isoprene and its possible subsequent physical separation and chemical conversion can be performed according to any method known to one of skill in the art and as described above.

In a particular embodiment, the method also comprises detecting isoprene which is present in the gaseous phase. The presence of isoprene in an environment of air or another gas, even in small amounts, can be detected by using various techniques and in particular by using gas chromatography systems with infrared or flame ionization detection, or by coupling with mass spectrometry.

When the process according to the invention is carried out in vivo by using an organism/microorganism providing the respective enzyme activities, the organism, preferably microorganism, is cultivated under suitable culture conditions allowing the occurrence of the enzymatic reaction. The specific culture conditions depend on the specific organism/microorganism employed but are well known to the person skilled in the art. The culture conditions are generally chosen in such a manner that they allow the expression of the genes encoding the enzymes for the respective reactions. Various methods are known to the person skilled in the art in order to improve and fine-tune the expression of certain genes at certain stages of the culture such as induction of gene expression by chemical inducers or by a temperature shift.

In another embodiment the organism employed in the method according to the invention is a plant. In principle any possible plant can be used, i.e. a monocotyledonous plant or a dicotyledonous plant. It is preferable to use a plant which can be cultivated on an agriculturally meaningful scale and which allows to produce large amounts of biomass. Examples are grasses like Lolium, cereals like rye, wheat, barley, oat, millet, maize, other starch storing plants like potato or sugar storing plants like sugar cane or sugar beet. Conceivable is also the use of tobacco or of vegetable plants such as tomato, pepper, cucumber, egg plant etc. Another possibility is the use of oil storing plants such as rape seed, olives etc. Also conceivable is the use of trees, in particular fast growing trees such as eucalyptus, poplar or rubber tree (Hevea brasiliensis).

In another embodiment, the method according to the invention is characterized by the conversion of a carbon source, such as glucose, into isoprenol followed by the conversion of isoprenol into isoprene according to any one of the above described Pathways A to F.

In another embodiment, the method according to the invention comprises the production of isoprene from atmospheric CO₂ or from CO₂ artificially added to the culture medium. In this case the method is implemented in an organism which is able to carry out photosynthesis, such as for example microalgae.

As described above, it is possible to use in the method according to the invention a (micro)organism which is genetically modified so as to contain a nucleic acid molecule encoding at least one of the enzymes as described above in connection with any one of the Pathways A to F. Such a nucleic acid molecule encoding an enzyme as described above can be used alone or as part of a vector. The nucleic acid molecules can further comprise expression control sequences operably linked to the polynucleotide comprised in the nucleic acid molecule. The term “operatively linked” or “operably linked”, as used throughout the present description, refers to a linkage between one or more expression control sequences and the coding region in the polynucleotide to be expressed in such a way that expression is achieved under conditions compatible with the expression control sequence.

Expression comprises transcription of the heterologous DNA sequence, preferably into a translatable mRNA. Regulatory elements ensuring expression in fungi as well as in bacteria, are well known to those skilled in the art. They encompass promoters, enhancers, termination signals, targeting signals and the like. Examples are given further below in connection with explanations concerning vectors.

Promoters for use in connection with the nucleic acid molecule may be homologous or heterologous with regard to its origin and/or with regard to the gene to be expressed. Suitable promoters are for instance promoters which lend themselves to constitutive expression. However, promoters which are only activated at a point in time determined by external influences can also be used. Artificial and/or chemically inducible promoters may be used in this context.

The vectors can further comprise expression control sequences operably linked to said polynucleotides contained in the vectors. These expression control sequences may be suited to ensure transcription and synthesis of a translatable RNA in bacteria or fungi. In addition, it is possible to insert different mutations into the polynucleotides by methods usual in molecular biology (see for instance Sambrook and Russell (2001), Molecular Cloning: A Laboratory Manual, CSH Press, Cold Spring Harbor, N.Y., USA), leading to the synthesis of polypeptides possibly having modified biological properties. The introduction of point mutations is conceivable at positions at which a modification of the amino acid sequence for instance influences the biological activity or the regulation of the polypeptide.

Moreover, mutants possessing a modified substrate or product specificity can be prepared. Preferably, such mutants show an increased activity. Furthermore, the introduction of mutations into the polynucleotides encoding an enzyme as defined above allows the gene expression rate and/or the activity of the enzymes encoded by said polynucleotides to be optimized.

For genetically modifying bacteria or fungi, the polynucleotides encoding an enzyme as defined above or parts of these molecules can be introduced into plasmids which permit mutagenesis or sequence modification by recombination of DNA sequences. Standard methods (see Sambrook and Russell (2001), Molecular Cloning: A Laboratory Manual, CSH Press, Cold Spring Harbor, N.Y., USA) allow base exchanges to be performed or natural or synthetic sequences to be added. DNA fragments can be connected to each other by applying adapters and linkers to the fragments. Moreover, engineering measures which provide suitable restriction sites or remove surplus DNA or restriction sites can be used. In those cases, in which insertions, deletions or substitutions are possible, in vitro mutagenesis, “primer repair”, restriction or ligation can be used. In general, a sequence analysis, restriction analysis and other methods of biochemistry and molecular biology are carried out as analysis methods. The polynucleotide introduced into a (micro)organism is expressed so as to lead to the production of a polypeptide having any of the activities described above in connection with Pathways A to F. An overview of different expression systems is for instance contained in Methods in Enzymology 153 (1987), 385-516, in Bitter et al. (Methods in Enzymology 153 (1987), 516-544) and in Sawers et al. (Applied Microbiology and Biotechnology 46 (1996), 1-9), Billman-Jacobe (Current Opinion in Biotechnology 7 (1996), 500-4), Hockney (Trends in Biotechnology 12 (1994), 456-463), Griffiths et al., (Methods in Molecular Biology 75 (1997), 427-440). An overview of yeast expression systems is for instance given by Hensing et al. (Antonie van Leuwenhoek 67 (1995), 261-279), Bussineau et al. (Developments in Biological Standardization 83 (1994), 13-19), Gellissen et al. (Antonie van Leuwenhoek 62 (1992), 79-93, Fleer (Current Opinion in Biotechnology 3 (1992), 486-496), Vedvick (Current Opinion in Biotechnology 2 (1991), 742-745) and Buckholz (Bio/Technology 9 (1991), 1067-1072).

Expression vectors have been widely described in the literature. As a rule, they contain not only a selection marker gene and a replication-origin ensuring replication in the host selected, but also a bacterial or viral promoter, and in most cases a termination signal for transcription. Between the promoter and the termination signal there is in general at least one restriction site or a polylinker which enables the insertion of a coding DNA sequence. The DNA sequence naturally controlling the transcription of the corresponding gene can be used as the promoter sequence, if it is active in the selected host organism. However, this sequence can also be exchanged for other promoter sequences. It is possible to use promoters ensuring constitutive expression of the gene and inducible promoters which permit a deliberate control of the expression of the gene. Bacterial and viral promoter sequences possessing these properties are described in detail in the literature. Regulatory sequences for the expression in microorganisms (for instance E. coli, S. cerevisiae) are sufficiently described in the literature. Promoters permitting a particularly high expression of a downstream sequence are for instance the T7 promoter (Studier et al., Methods in Enzymology 185 (1990), 60-89), lacUV5, trp, trp-lacUV5 (DeBoer et al., in Rodriguez and Chamberlin (Eds), Promoters, Structure and Function; Praeger, N.Y., (1982), 462-481; DeBoer et al., Proc. Natl. Acad. Sci. USA (1983), 21-25), Ip1, rac (Boros et al., Gene 42 (1986), 97-100). Inducible promoters are preferably used for the synthesis of polypeptides. These promoters often lead to higher polypeptide yields than do constitutive promoters. In order to obtain an optimum amount of polypeptide, a two-stage process is often used. First, the host cells are cultured under optimum conditions up to a relatively high cell density. In the second step, transcription is induced depending on the type of promoter used. In this regard, a tac promoter is particularly suitable which can be induced by lactose or IPTG (=isopropyl-R-D-thiogalactopyranoside) (deBoer et al., Proc. Natl. Acad. Sci. USA 80 (1983), 21-25). Termination signals for transcription are also described in the literature.

The transformation of the host cell with a polynucleotide or vector according to the invention can be carried out by standard methods, as for instance described in Sambrook and Russell (2001), Molecular Cloning: A Laboratory Manual, CSH Press, Cold Spring Harbor, N.Y., USA; Methods in Yeast Genetics, A Laboratory Course Manual, Cold Spring Harbor Laboratory Press, 1990. The host cell is cultured in nutrient media meeting the requirements of the particular host cell used, in particular in respect of the pH value, temperature, salt concentration, aeration, antibiotics, vitamins, trace elements etc.

The present invention also relates to an organism, preferably a microorganism, which is able to express the enzymes required for the conversion of isoprenol into isoprene according to any of the Pathways A to F of the method of the invention as described above and which is able to convert isoprenol into isoprene.

Thus, the present invention also relates to a (micro)organism which expresses
A) (a) a hydroxyethylthiazole kinase (EC 2.7.1.50); and
   (b) a terpene synthase, e.g. an isoprene synthase (EC 4.2.3.27) and/or a monoterpene synthase and/or an alpha-farnesene synthases (EC 4.2.3.46) and/or an beta-farnesene synthase (EC 4.2.3.47) and/or a myrcene/(E)-beta-ocimene synthase (EC 4.2.3.15) and/or a pinene synthase (EC 4.2.3.14);
      or
B) (a) a hydroxyethylthiazole kinase (EC 2.7.1.50); and
   (b) an isopentenyl-diphosphate DELTA isomerase (EC 5.3.3.2); and
   (c) a terpene synthase, e.g. an isoprene synthase (EC 4.2.3.27) and/or a monoterpene synthase and/or an alpha-farnesene synthases (EC 4.2.3.46) and/or an beta-farnesene synthase (EC 4.2.3.47) and/or a myrcene/(E)-beta-ocimene synthase (EC 4.2.3.15) and/or a pinene synthase (EC 4.2.3.14);
or
D) (a) a sulfotransferase (EC 2.8.2); and
   (b) a terpene synthase, e.g. an isoprene synthase (EC 4.2.3.27) and/or a monoterpene synthase and/or an alpha-farnesene synthases (EC 4.2.3.46) and/or an beta-farnesene synthase (EC 4.2.3.47) and/or a myrcene/(E)-beta-ocimene synthase (EC 4.2.3.15) and/or a pinene synthase (EC 4.2.3.14) or a retinol sulfotransferase/dehydratase;
or
E) (a) a sulfotransferase (EC 2.8.2); and
   (b) an isopentenyl-diphosphate DELTA isomerase (EC 5.3.3.2); and
   (c) a terpene synthase, e.g. an isoprene synthase (EC 4.2.3.27) and/or a monoterpene synthase and/or an alpha-farnesene synthases (EC 4.2.3.46) and/or an beta-farnesene synthase (EC 4.2.3.47) and/or a myrcene/(E)-beta-ocimene synthase (EC 4.2.3.15) and/or a pinene synthase (EC 4.2.3.14) or a retinol sulfotransferase/dehydratase;
      and which is capable of converting isoprenol into isoprene.

In one embodiment an organism according to the present invention is a recombinant organism in the sense that it is genetically modified due to the introduction of at least one nucleic acid molecule encoding at least one of the above mentioned enzymes.

Preferably such a nucleic acid molecule is heterologous with regard to the organism which means that it does not naturally occur in said organism.

The microorganism is preferably a bacterium, a yeast or a fungus. In another preferred embodiment the organism is a plant or non-human animal. As regards other preferred embodiments, the same applies as has been set forth above in connection with the method according to the invention.

The present invention also relates to a composition comprising
A) (a) a hydroxyethylthiazole kinase (EC 2.7.1.50); and
   (b) a terpene synthase, e.g. an isoprene synthase (EC 4.2.3.27) and/or a monoterpene synthase and/or an alpha-farnesene synthases (EC 4.2.3.46) and/or an beta-farnesene synthase (EC 4.2.3.47) and/or a myrcene/(E)-beta-ocimene synthase (EC 4.2.3.15) and/or a pinene synthase (EC 4.2.3.14);
or
B) (a) a hydroxyethylthiazole kinase (EC 2.7.1.50); and
   (b) an isopentenyl-diphosphate DELTA isomerase (EC 5.3.3.2); and
   (c) a terpene synthase, e.g. an isoprene synthase (EC 4.2.3.27) and/or a monoterpene synthase and/or an alpha-farnesene synthases (EC 4.2.3.46) and/or an beta-farnesene synthase (EC 4.2.3.47) and/or a myrcene/(E)-beta-ocimene synthase (EC 4.2.3.15) and/or a pinene synthase (EC 4.2.3.14);
or
D) (a) a sulfotransferase (EC 2.8.2); and
   (b) a terpene synthase, e.g. an isoprene synthase (EC 4.2.3.27) and/or a monoterpene synthase and/or an alpha-farnesene synthases (EC 4.2.3.46) and/or an beta-farnesene synthase (EC 4.2.3.47) and/or a myrcene/(E)-beta-ocimene synthase (EC 4.2.3.15) and/or a pinene synthase (EC 4.2.3.14) or a retinol sulfotransferase/dehydratase;
or
E) (a) a sulfotransferase (EC 2.8.2); and
   (b) an isopentenyl-diphosphate DELTA isomerase (EC 5.3.3.2); and
   (c) a terpene synthase, e.g. an isoprene synthase (EC 4.2.3.27) and/or a monoterpene synthase and/or an alpha-farnesene synthases (EC 4.2.3.46) and/or an beta-farnesene synthase (EC 4.2.3.47) and/or a myrcene/(E)-beta-ocimene synthase (EC 4.2.3.15) and/or a pinene synthase (EC 4.2.3.14) or a retinol sulfotransferase/dehydratase.

Such a composition may also comprise isoprenol. As regards preferred embodiments, the same applies as has been set forth above in connection with the method according to the invention.

The present invention also relates to the use of a combination of enzymes comprising:
A) (a) a hydroxyethylthiazole kinase (EC 2.7.1.50); and
   (b) a terpene synthase, e.g. an isoprene synthase (EC 4.2.3.27) and/or a monoterpene synthase and/or an alpha-farnesene synthases (EC 4.2.3.46) and/or an beta-farnesene synthase (EC 4.2.3.47) and/or a myrcene/(E)-beta-ocimene synthase (EC 4.2.3.15) and/or a pinene synthase (EC 4.2.3.14);
or
B) (a) a hydroxyethylthiazole kinase (EC 2.7.1.50); and
   (b) an isopentenyl-diphosphate DELTA isomerase (EC 5.3.3.2); and
   (c) a terpene synthase, e.g. an isoprene synthase (EC 4.2.3.27) and/or a monoterpene synthase and/or an alpha-farnesene synthases (EC 4.2.3.46) and/or an beta-farnesene synthase (EC 4.2.3.47) and/or a myrcene/(E)-beta-ocimene synthase (EC 4.2.3.15) and/or a pinene synthase (EC 4.2.3.14);
or
D) (a) a sulfotransferase (EC 2.8.2); and
   (b) a terpene synthase, e.g. an isoprene synthase (EC 4.2.3.27) and/or a monoterpene synthase and/or an alpha-farnesene synthases (EC 4.2.3.46) and/or an beta-farnesene synthase (EC 4.2.3.47) and/or a myrcene/(E)-beta-ocimene synthase (EC 4.2.3.15) and/or a pinene synthase (EC 4.2.3.14) or a retinol sulfotransferase/dehydratase;
or
E) (a) a sulfotransferase (EC 2.8.2); and
   (b) an isopentenyl-diphosphate DELTA isomerase (EC 5.3.3.2); and
   (c) a terpene synthase, e.g. an isoprene synthase (EC 4.2.3.27) and/or a monoterpene synthase and/or an alpha-farnesene synthases (EC 4.2.3.46) and/or an beta-farnesene synthase (EC 4.2.3.47) and/or a myrcene/(E)-beta-ocimene synthase (EC 4.2.3.15)) and/or a pinene synthase (EC 4.2.3.14) or a retinol sulfotransferase/dehydratase
      for the production of isoprene from isoprenol.

The present invention also relates to the use of
(i) a terpene synthase, e.g. an isoprene synthase (EC 4.2.3.27) and/or an alpha-farnesene synthases (EC 4.2.3.46) and/or an beta-farnesene synthase (EC 4.2.3.47) and/or a myrcene/(E)-beta-ocimene synthase (EC 4.2.3.15) and/or a pinene synthase (EC 4.2.3.14); or
(ii) a sulfotransferase (EC 2.8.2)
   for the production of isoprene from isoprenol.

Other aspects and advantages of the invention will be described in the following examples, which are given for purposes of illustration and not by way of limitation. Each publication, patent, patent application or other document cited in this application is hereby incorporated in its entirety for all purposes to the same extent as if each were individually indicated to be incorporated by reference for all purposes in the specification directly adjacent the citation.

### EXAMPLES

### Example 1

### Cloning, Expression and Purification of Enzymes

Cloning, bacterial cultures and expression of proteins

The genes encoding the enzymes of interest were cloned in the pET 25b(+) vector (Novagen). Nucleotide sequences encoding a chloroplast transit peptide in plant terpene synthases were removed, resulting in DNA sequences encoding the mature proteins only. A stretch of 6 histidine codons was inserted after the methionine initiation codon to provide an affinity tag for purification. Competent E. coli BL21(DE3) cells (Novagen) were transformed with this vector by heat shock. The transformed cells were grown with shaking (160 rpm) on ZYM-5052 auto-induction medium (Studier FW, Prot. Exp. Pur. 41, (2005), 207-234) for 6 h at 37° C. and protein expression was continued at 28° C. or 20° C. overnight (approximately 16 h). The cells were collected by centrifugation at 4° C., 10,000 rpm for 20 min and the pellets were frozen at −80° C.

Protein Purification and Concentration.

The pellets from 200 ml of culture cells were thawed on ice and resuspended in 5 ml of Na₂HPO₄ pH 8 containing 300 mM NaCl, 5 mM MgCl₂ and 1 mM DTT. Twenty microliters of lysonase (Novagen) were added. Cells were incubated 10 minutes at room temperature and then returned to ice for 20 minutes. Cell lysis was completed by sonication for 3×15 seconds. The bacterial extracts were then clarified by centrifugation at 4° C., 10,000 rpm for 20 min. The clarified bacterial lysates were loaded on PROTINO-1000 Ni-TED column (Macherey-Nagel) allowing adsorption of 6-His tagged proteins. Columns were washed and the enzymes of interest were eluted with 4 ml of 50 mM Na₂HPO₄ pH 8 containing 300 mM NaCl, 5 mM MgCl₂, 1 mM DTT, 250 mM imidazole. Eluates were then concentrated and desalted on Amicon Ultra-4 10 kDa filter unit (Millipore) and resuspended in 0.25 ml 50 mM Tris-HCl pH 7.4 containing 0.5 mM DTT and 5 mM MgCl₂. Protein concentrations were quantified according to the Bradford method. The purity of proteins thus purified varied from 70% to 90%.

### Example 2

### Characterization of Isoprenol Phosphorylation Activity

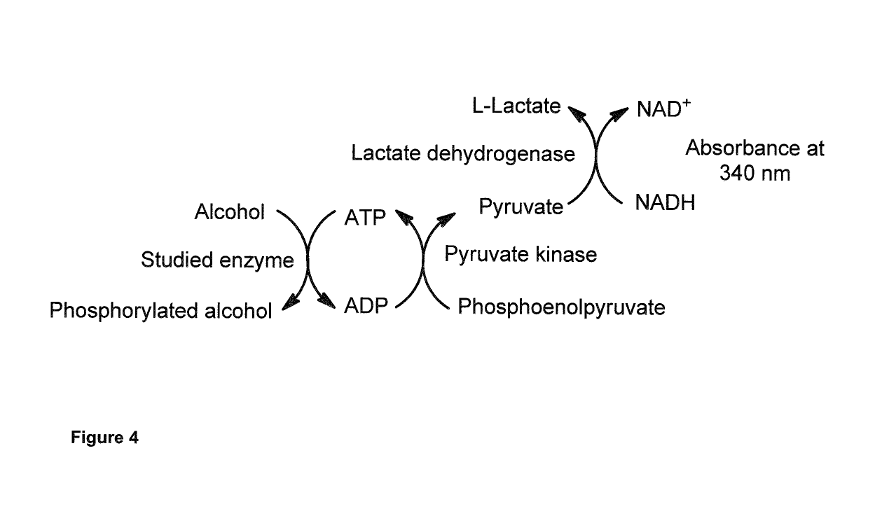

The release of ADP that is associated with isoprenol phosphorylation was quantified using the pyruvate kinase/lactate dehydrogenase coupled assay (FIG. 4). The purified 4-methyl-5-(2-hydroxyethyl)thiazole kinases from Escherichia coli, Bacillus subtilis, Rhizobium leguminosarum were thus evaluated for their ability to phosphorylate isoprenol.

The studied enzymatic reaction was carried out under the following conditions at 37° C.:

50 mM Tris-HCl pH 7.5

10 mM MgCl₂

100 mM KCl

5 mM ATP

0.4 mM NADH

1 mM Phosphoenolpyruvate

3 U/ml Lactate dehydrogenase

1.5 U/ml Pyruvate kinase

0-40 mM isoprenol

The pH was adjusted to 7.5

Each assay was started by the addition of the enzyme at a final concentration 0.025 mg/ml and the decrease of NADH was monitored by following its absorbance at 340 nm.

Assays with hydroxyethylthiazole kinase gave rise to a reproducible and significant increase in ADP production in the presence of isoprenol. The kinetic parameters of isoprenol phosphorylation for several kinases are shown in the following Table 2.

TABLE 2

| Kinase | K_{M}, mM | k_{cat}, s⁻¹ | k_{cat}/K_{M}, mM⁻¹ s⁻¹ |
|---|---|---|---|
| E. coli | 7.5 | 1.0 | 0.13 |
| R. leguminosarum | 10 | 0.8 | 0.08 |
| B. subtilis | 3 | 0.03 | 0.01 |

### Example 3

### Analysis of Isoprenol Phosphorylation Reaction by Mass Spectrometry

The studied enzymatic reactions were carried out under the following conditions:

50 mM Tris-HCl pH 8

200 mM isoprenol

5 mM MgCl₂

50 mM ATP

3 mM 2-Mercaptoethanol

5 mg/ml purified hydroxythiazole kinase from R. leguminosarum

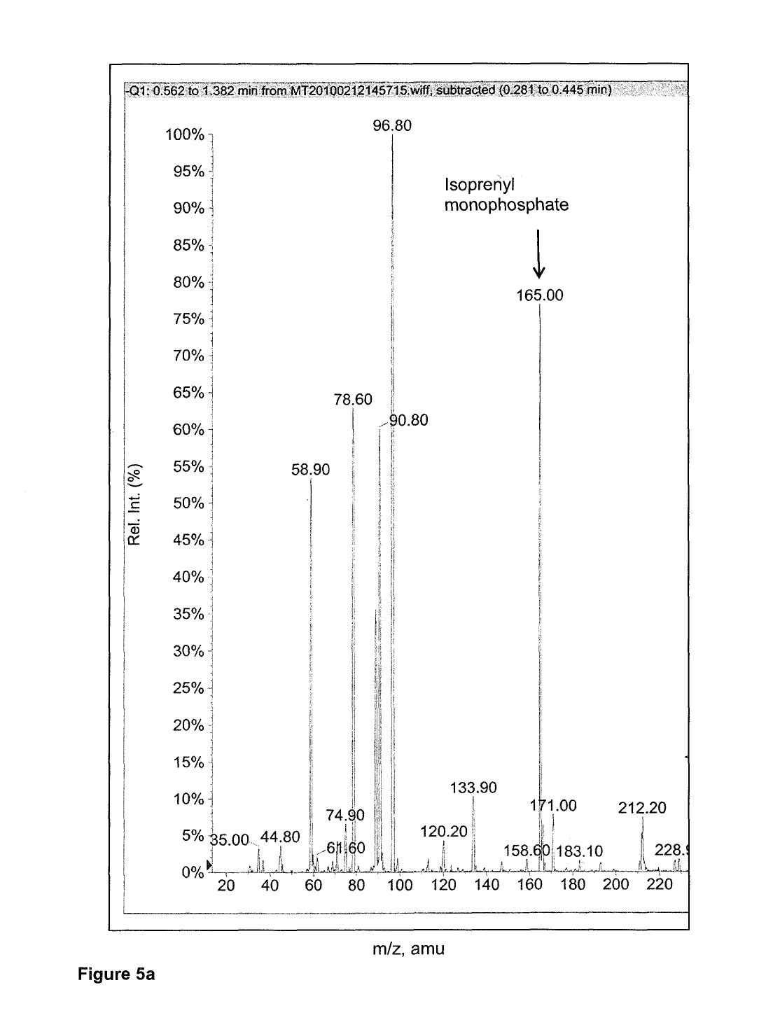

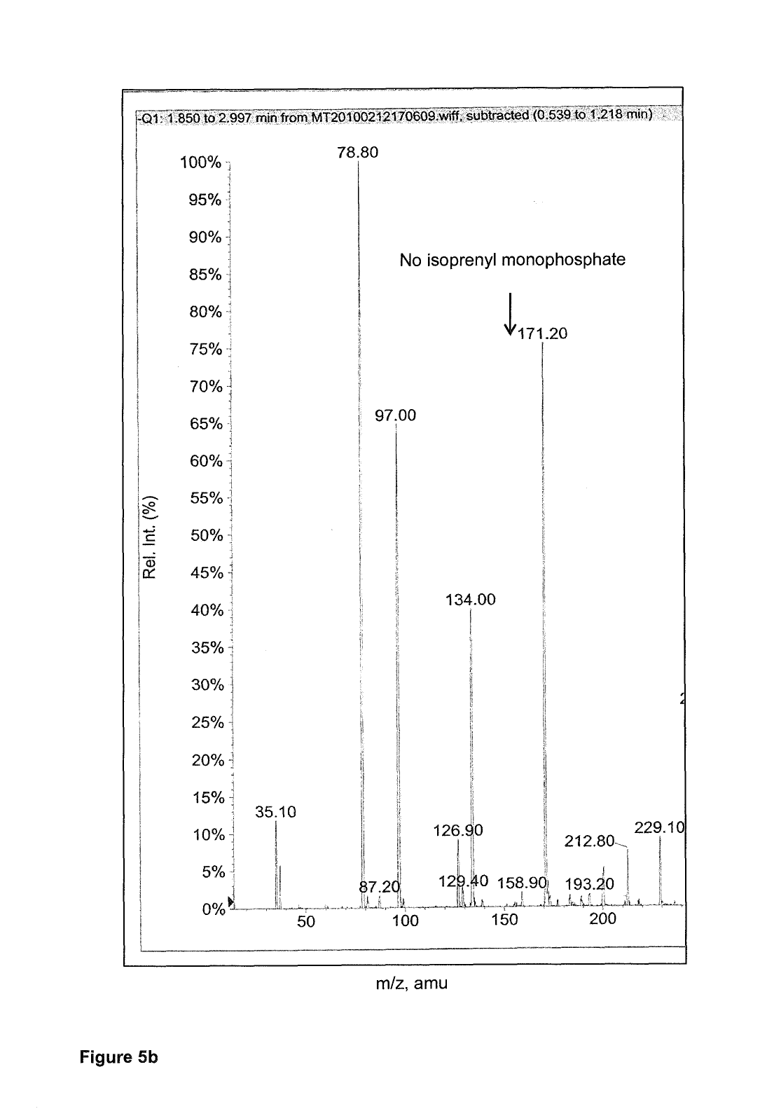

Control reactions were set up either without enzyme, or without substrate. Following incubation, assays were analyzed by mass spectrometry (MS) using a negative ion mode. Typically, an aliquot of 100 μl reaction was removed, centrifuged and the supernatant was transferred to a clean vial. The product was then diluted 1:5 (20%, vol/vol) with methanol. An aliquot of 5 μl was directly injected into mass spectrometer. Detection was performed by a PE SCIEX API 2000 quadrupole spectrometer interfaced to an electrospray ionisation (ESI) source. MS analysis showed the presence of an [M-H]⁻ ion at m/z=165.00, corresponding to isoprenyl monophosphate (3-methylbut-3-enyl hydrogen phosphate), in the complete enzymatic assay (FIG. 5a), but not in the controls (FIG. 5b).

### Example 4

### Characterization of Prenol Phosphorylation Activity

The purified 4-methyl-5-(2-hydroxyethyl) thiazole kinases from Escherichia coli, Bacillus subtilis, Rhizobium leguminosarum were evaluated for their ability to phosphorylate prenol. The release of ADP associated to prenol phosphorylation was quantified using the pyruvate kinase/lactate dehydrogenase coupled assay (FIG. 4). The studied enzymatic reaction was carried out according to the protocol described in example 2.

Each assay was started by the addition of the enzyme at a final concentration 0.5 mg/ml and the decrease of NADH was monitored by following its absorbance at 340 nm.

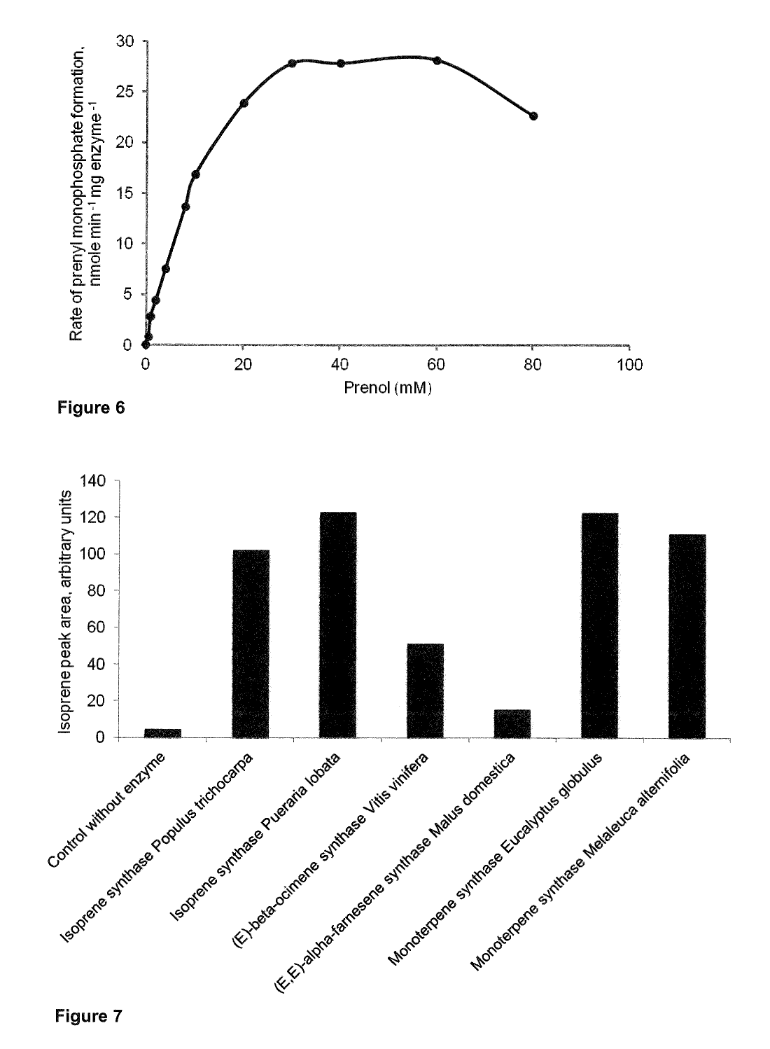

Assays with hydroxyethylthiazole kinase gave rise to a reproducible and significant increase in ADP production in the presence of prenol. FIG. 6 shows an example of a Michaelis-Menten plot corresponding to the data collected for R. leguminosarum enzyme. The kinetic parameters of prenol phosphorylation for several kinases are shown in the following Table 3.

TABLE 3

| Kinase | K_{M}, mM | k_{cat}, s⁻¹ | k_{cat}/K_{M}, mM⁻¹ s⁻¹ |
|---|---|---|---|
| E. coli | 3 | 0.05 | 0.016 |
| R. leguminosarum | 10 | 0.01 | 0.001 |
| B. subtilis | 3 | 0.006 | 0.002 |

### Example 5

### Screening for Isoprene Production from Isoprenyl Monophosphate with Purified Terpene Synthases

The enzymatic assays were carried out under the following conditions at 37° C.:

50 mM Tris-HCl pH7.5

20 mM MgCl₂

1 mM DTT

10 mM isoprenyl monophosphate (Sigma)

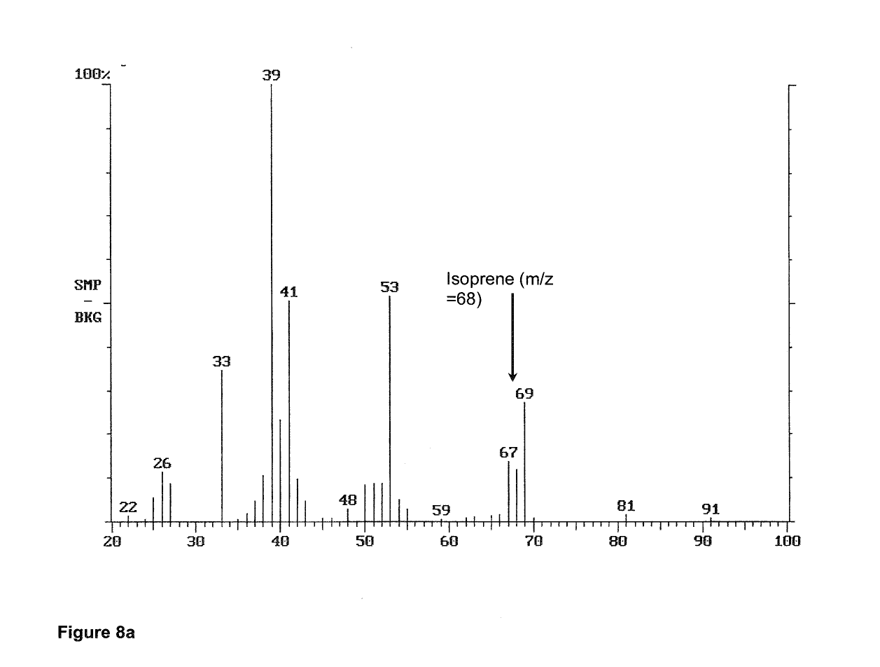

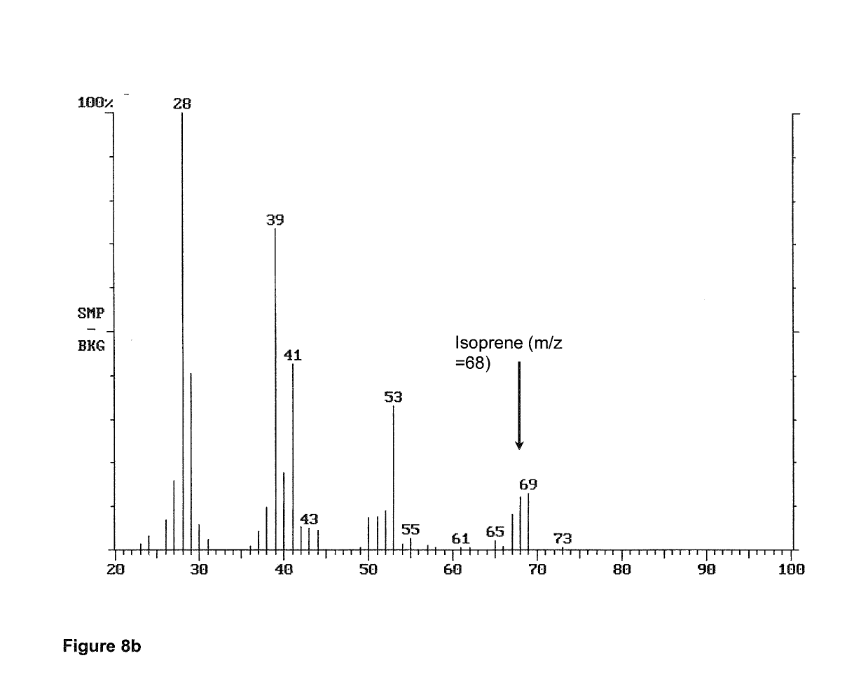

2.5 mg of the terpene synthase was added to 0.5 ml of reaction mixture. An enzyme-free control reaction was carried out in parallel. Assays were incubated at 37° C. for 17 h in a 1.5 ml sealed glass vial (Interchim) with shaking. One ml of the headspace phase was then collected and injected into a gas chromatograph Varian 430-GC chromatograph equipped with a flame ionization detector (FID). Nitrogen was used as carrier gas with a flow rate of 30 mL/min. Volatile compounds were chromatographically separated on Rtx-1 column (Restek) using an isothermal mode at 100° C. The enzymatic reaction product was identified by direct comparison with isoprene standard (Sigma). Under these GC conditions, the retention time for isoprene was 3.47 min. A significant production of isoprene was observed with several purified terpene synthases (FIG. 7). Gas chromatography-mass spectrometry (GC-MS) was then used to confirm the identity of the product detected by gas chromatography with flame ionization. The samples were analyzed on a Varian 3400Cx gas chromatograph equipped with Varian Saturn 3 mass selective detector. A mass spectrum of isoprene obtained by enzymatic conversion of isoprenyl monophosphate was similar to the one of commercial isoprene (FIG. 8).

### Example 6

### Kinetic parameters of isoprene production from isoprenyl monophosphate

Kinetic parameters of isoprene production were evaluated in the following conditions:

50 mM Tris-HCl pH7.5

20 mM MgCl₂

1 mM DTT

4 mg/ml monoterpene synthase

0-150 mM isoprenyl monophosphate

The assays were incubated at 37° C. for 18 h in a sealed glass vial (Interchim) with shaking. Isoprene production was analyzed using the GC/FID procedure described in example 5 and quantified using commercial isoprene.

The K_{M} and k_{cat} values for purified monoterpene synthase from E. globulus were about 40 mM and 1.6×10⁻⁵ s⁻¹, respectively.

### Example 7

### Screening for Isoprene Production from Prenyl Monophosphate with Purified Terpene Synthases

The enzymatic assays were carried out under the following conditions at 37° C.:

50 mM Tris-HCl pH7.5

20 mM MgCl₂

1 mM DTT

5 mM prenyl monophosphate (Sigma)

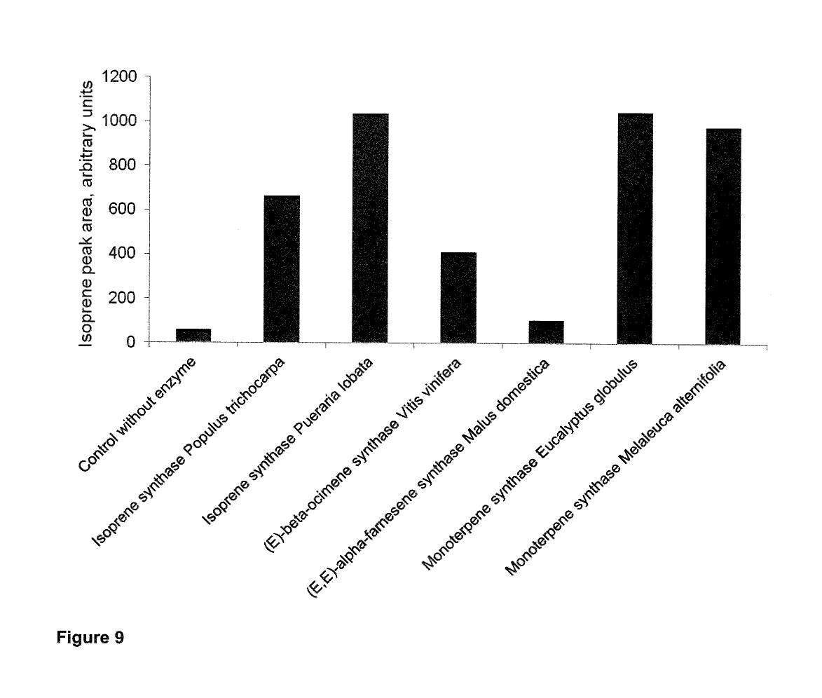

1.25 mg of the terpene synthase to be tested was added to 0.5 ml of reaction mixture. An enzyme-free control reaction was carried out in parallel. Assays were incubated at 37° C. for 22 h in a 1.5 ml sealed glass vial (Interchim) with shaking. Isoprene analysis was performed according to the procedure described in example 5. Several purified terpene synthases were able to catalyze the studied reaction (FIG. 9).

### Example 8

### Kinetic Parameters of Isoprene Production from Prenyl Monophosphate

The enzymatic assays were carried out under the following conditions:

50 mM Tris-HCl pH 7.5

20 mM MgCl₂

1 mM DTT

2 mg/ml monoterpene synthase

0-150 mM prenyl monophosphate (Sigma)

The assays were incubated at 37° C. for 18 h in a sealed vial (Interchim) with shaking. Isoprene production was analyzed by GC/FID procedure described in Example 5 and quantified using commercial isoprene. The purified monoterpene synthase from E. globulus exhibited K_{M} and k_{cat} values of 60 mM and 5.6×10⁻⁴ s⁻¹, respectively.

### Example 9

### Isomerization of Isoprenyl Monophosphate to Prenyl Monophosphate by Purified Isopentenyl Diphosphate Delta Isomerase

The enzymatic assays are carried out under the following conditions:

50 mM Tris-HCl pH7.5

20 mM MgCl₂

1 mM DTT

5 mg/ml purified isomerase

50 mM isoprenyl monophosphate

The assays are incubated at 37° C. with shaking. Control reactions are performed either without enzyme, or without substrate. At the end of the incubation period, 80 μl of samples are removed, centrifuged and the supernatant is transferred to a clean vial. An aliquot of 20 μl is analyzed by HPLC/UV (Agilent 1260 Infinity).

### Example 10

### Isomerization of Isoprenol to Prenol by Purified Isopentenyl Diphosphate Delta Isomerase

The enzymatic assays are carried out under the following conditions:

50 mM Tris-HCl pH7.5

20 mM MgCl₂

1 mM DTT

5 mg/ml purified isomerase

50 mM isoprenol

The assays are incubated at 37° C. with shaking. Control reactions are performed either without enzyme, or without substrate. At the end of the incubation samples are analyzed by HPLC or by Gas Chromatography (GC) for measurement of prenol production.

### Example 11

### Analysis of Isoprenol Sulfotransferase Assay by Mass Spectrometry

The studied enzymatic reactions are carried out under the following conditions:

50 mM Tris-HCl pH 7.5

50 mM Isoprenol

50 mM PAPS

3 mM 2-Mercaptoethanol

5 mg/ml purified sulfotransferase

Control reactions are set up either without enzyme, or without substrate. Following incubation, assays are analyzed by mass spectrometry (MS) using a negative ion mode. Typically, an aliquot of 200 μl reaction is removed, centrifuged and the supernatant is transferred to a clean vial. An aliquot of 5-100 μl is then directly injected into mass spectrometer.

### Example 12

### Analysis of prenol sulfotransferase assay by mass spectrometry

The studied enzymatic reactions are carried out under the following conditions:

50 mM Tris-HCl pH 7.5

50 mM Prenol

50 mM PAPS

3 mM 2-Mercaptoethanol

5 mg/ml purified sulfotransferase

Control reactions are set up either without enzyme, or without substrate. Following incubation, assays are analyzed by mass spectrometry (MS) using a negative ion mode. Typically, an aliquot of 200 μl reaction is removed, centrifuged and the supernatant is transferred to a clean vial. An aliquot of 5-100 μl is then directly injected into mass spectrometer.

### Example 13

### Screening for Isoprene Production from Prenyl Sulfate with Purified Terpene Synthases

The enzymatic assays are carried out under the following conditions at 37° C.:

50 mM Tris-HCl pH7.5

20 mM MgCl₂

1 mM DTT

0-50 mM prenyl sulfate

2 mg of the terpene synthase to be tested is added to 0.5 ml of reaction mixture. An enzyme-free control reaction is carried out in parallel. Assays are incubated at 37° C. for 24-72 h in a 1.5 ml sealed glass vial (Interchim) with shaking. Isoprene analysis is performed according to the procedure described in Example 5.

### Example 14

### Screening for Isoprene Production from Isoprenyl Sulfate with Purified Terpene Synthases

The enzymatic assays were carried out under the following conditions at 37° C.:

50 mM Tris-HCl pH7.5

20 mM MgCl₂

1 mM DTT

0-50 mM isoprenyl sulfate

2 mg of the terpene synthase to be tested is added to 0.5 ml of reaction mixture. An enzyme-free control reaction is carried out in parallel. Assays are incubated at 37° C. for 24-72 h in a 1.5 ml sealed glass vial (Interchim) with shaking. Isoprene analysis is performed according to the procedure described in Example 5.

1. A method of producing isoprene from isoprenol comprising enzymatically converting isoprenol into isoprenyl monophosphate and then enzymatically converting isoprenyl monophosphate into isoprene.

2. The method of claim 1 wherein the enzymatic conversion of isoprenol into isoprenyl monophosphate is achieved by the use of a hydroxyethylthiazole kinase (EC 2.7.1.50).

3. The method of claim 1 wherein isoprenyl monophosphate is directly enzymatically converted into isoprene (Pathway A).

4. The method of claim 3 wherein the enzymatic conversion of isoprenyl monophosphate into isoprene is achieved by the use of a terpene synthase.

5. The method of claim 4 wherein the terpene synthase is selected from an isoprene synthase (EC 4.2.3.27), a monoterpene synthase, an alpha-farnesene synthases (EC 4.2.3.46), an beta-farnesene synthase (EC 4.2.3.47,) a myrcene/(E)-beta-ocimene synthase (EC 4.2.3.15) or a pinene synthase (EC 4.2.3.14).

6. The method of claim 1 wherein isoprenyl monophosphate is converted enzymatically into prenyl monophosphate by an isomerisation reaction and prenyl monophosphate is then converted into isoprene by a dephosphorylation reaction (Pathway B).

7. The method of claim 6 wherein the enzymatic conversion of isoprenyl monophosphate into prenyl monophosphate is achieved by the use of an isopentenyl-diphosphate DELTA isomerase (EC 5.3.3.2) and wherein the enzymatic conversion of prenyl monophosphate into isoprene is achieved by the use of a terpene synthase.

8. A method of producing isoprene from isoprenol comprising enzymatically converting isoprenol into prenol by an isomerisation reaction, and then enzymatically converting prenol into prenyl monophosphate by a phosphorylation reaction and then enzymatically converting prenyl monophosphate into isoprene (Pathway C).

9. The method of claim 8 wherein the enzymatic conversion of isoprenol into prenol is achieved by the use of an isopentenyl-diphosphate DELTA isomerase (EC 5.3.3.2).

10. The method of claim 8 wherein the enzymatic conversion of prenol into prenyl monophosphate is achieved by the use of a hydroxyethylthiazole kinase (EC 2.7.1.50).

11. The method of claim 8 wherein prenyl monophosphate is directly converted into isoprene by a dephosphorylation reaction.

12. The method of claim 11 wherein the enzymatic conversion of prenyl monophosphate into isoprene is achieved by the use of a terpene synthase.

13. A method of producing isoprene from isoprenol comprising enzymatically converting isoprenol into isoprenyl sulfate and then enzymatically converting isoprenyl sulfate into isoprene.

14. The method of claim 13 wherein the enzymatic conversion of isoprenol into isoprenyl sulfate is achieved by the use of a sulfotransferase (EC 2.8.2).

15. The method of claim 13 wherein isoprenyl sulfate is directly converted into isoprene (Pathway D) by a thermal or enzymatic desulfurylation reaction.

16. The method of claim 15 wherein the enzymatic conversion of isoprenyl sulfate into isoprene is achieved by the use of a terpene synthase or a retinol sulfotransferase/dehydratase.

17. The method of claim 16, wherein the terpene synthase is selected from an isoprene synthase (EC 4.2.3.27), a monoterpene synthase, an alpha-farnesene synthases (EC 4.2.3.46), an beta-farnesene synthase (EC 4.2.3.47,) a myrcene/(E)-beta-ocimene synthase (EC 4.2.3.15) or a pinene synthase (EC 4.2.3.14).

18. The method of claim 13 wherein isoprenyl sulfate is converted enzymatically into prenyl sulfate by an isomerisation reaction and prenyl sulfate is then converted into isoprene by a thermal or enzymatic desulfurylation reaction (Pathway E).

19. The method of claim 18 wherein the enzymatic conversion of isoprenyl sulfate into prenyl sulfate is achieved by the use of an isopentenyl-diphosphate DELTA isomerase (EC 5.3.3.2) and wherein the enzymatic conversion of prenyl sulfate into isoprene is achieved by the use of a terpene synthase or a retinol sulfotransferase/dehydratase.

20. A method of producing isoprene from isoprenol comprising enzymatically converting isoprenol into prenol by an isomerisation reaction, then enzymatically converting prenol into prenyl sulfate by a sulfurylation reaction and then enzymatically converting prenyl sulfate into isoprene (Pathway F).

21. The method of claim 20 wherein the enzymatic conversion of isoprenol into prenol is achieved by the use of an isopentenyl-diphosphate DELTA isomerase (EC 5.3.3.2).

22. The method of claim 20 wherein the enzymatic conversion of prenol into prenyl sulfate is achieved by the use of a sulfotransferase (EC 2.8.2).

23. The method of claim 20 wherein prenyl sulfate is directly converted into isoprene by a thermal or enzymatic desulfurylation reaction.

24. The method of claim 23 wherein the enzymatic conversion of prenyl sulfate into isoprene is achieved by the use of a terpene synthase or a retinol sulfotransferase/dehydratase.

25. A microorganism or a plant which expresses
A) (a) a hydroxyethylthiazole kinase (EC 2.7.1.50); and
(b) a monoterpene synthase, an alpha-farnesene synthases (EC 4.2.3.46), an beta-farnesene synthase (EC 4.2.3.47), a myrcene/(E)-beta-ocimene synthase (EC 4.2.3.15), or a pinene synthase (EC 4.2.3.14);
or
B) (a) a hydroxyethylthiazole kinase (EC 2.7.1.50); and
(b) an isopentenyl-diphosphate DELTA isomerase (EC 5.3.3.2); and
(c) a monoterpene synthase, an alpha-farnesene synthases (EC 4.2.3.46), a beta-farnesene synthase (EC 4.2.3.47), a myrcene/(E)-beta-ocimene synthase (EC 4.2.3.15), a pinene synthase (EC 4.2.3.14);
or
D) (a) a sulfotransferase (EC 2.8.2); and
(b) a monoterpene synthase, an alpha-farnesene synthases (EC 4.2.3.46), a beta-farnesene synthase (EC 4.2.3.47), a myrcene/(E)-beta-ocimene synthase (EC 4.2.3.15), a pinene synthase (EC 4.2.3.14), or a retinol sulfotransferase/dehydratase;
or
E) (a) a sulfotransferase (EC 2.8.2); and
(b) an isopentenyl-diphosphate DELTA isomerase (EC 5.3.3.2); and
(c) a monoterpene synthase, an alpha-farnesene synthases (EC 4.2.3.46), a beta-farnesene synthase (EC 4.2.3.47), a myrcene/(E)-beta-ocimene synthase (EC 4.2.3.15), a pinene synthase (EC 4.2.3.14), or a retinol sulfotransferase/dehydratase;
and which is capable of converting isoprenol into isoprene.

26. A composition comprising the microorganism or plant of claim 25 and, optionally, isoprenol.

27. A composition comprising
(A) (a) a hydroxyethylthiazole kinase (EC 2.7.1.50); and
(b) a monoterpene synthase, an alpha-farnesene synthases (EC 4.2.3.46), a beta-farnesene synthase (EC 4.2.3.47), a myrcene/(E)-beta-ocimene synthase (EC 4.2.3.15), a pinene synthase (EC 4.2.3.14);
or
B) (a) a hydroxyethylthiazole kinase (EC 2.7.1.50); and
(b) an isopentenyl-diphosphate DELTA isomerase (EC 5.3.3.2); and
(c) a monoterpene synthase, an alpha-farnesene synthases (EC 4.2.3.46), a beta-farnesene synthase (EC 4.2.3.47), a myrcene/(E)-beta-ocimene synthase (EC 4.2.3.15), a pinene synthase (EC 4.2.3.14);
D) (a) a sulfotransferase (EC 2.8.2); and
(b) a monoterpene synthase, an alpha-farnesene synthases (EC 4.2.3.46), a beta-farnesene synthase (EC 4.2.3.47), a myrcene/(E)-beta-ocimene synthase (EC 4.2.3.15), a pinene synthase (EC 4.2.3.14), or a retinol sulfotransferase/dehydratase;
or
E) (a) a sulfotransferase (EC 2.8.2); and
(b) an isopentenyl-diphosphate DELTA isomerase (EC 5.3.3.2); and
(c) a monoterpene synthase, an alpha-farnesene synthases (EC 4.2.3.46), a beta-farnesene synthase (EC 4.2.3.47), a myrcene/(E)-beta-ocimene synthase (EC 4.2.3.15), a pinene synthase (EC 4.2.3.14), or a retinol sulfotransferase/dehydratase.

28. The composition of claim 27 further comprising isoprenol.

29. A method of producing isoprene from isoprenol, wherein the method comprises converting isoprenol into isoprene using a combination of enzymes selected from:
A) (a) a hydroxyethylthiazole kinase (EC 2.7.1.50); and
(b) a terpene synthase, an isoprene synthase (EC 4.2.3.27), a monoterpene synthase, an alpha-farnesene synthases (EC 4.2.3.46), an beta-farnesene synthase (EC 4.2.3.47), a myrcene/(E)-beta-ocimene synthase (EC 4.2.3.15), a pinene synthase (EC 4.2.3.14);
or
B) (a) a hydroxyethylthiazole kinase (EC 2.7.1.50); and
(b) an isopentenyl-diphosphate DELTA isomerase (EC 5.3.3.2); and
(c) a terpene synthase, an isoprene synthase (EC 4.2.3.27), a monoterpene synthase, an alpha-farnesene synthases (EC 4.2.3.46), an beta-farnesene synthase (EC 4.2.3.47), a myrcene/(E)-beta-ocimene synthase (EC 4.2.3.15), a pinene synthase (EC 4.2.3.14);
or
D) (a) a sulfotransferase (EC 2.8.2); and
(b) a terpene synthase, an isoprene synthase (EC 4.2.3.27), a monoterpene synthase, an alpha-farnesene synthases (EC 4.2.3.46), an beta-farnesene synthase (EC 4.2.3.47), a myrcene/(E)-beta-ocimene synthase (EC 4.2.3.15), a pinene synthase (EC 4.2.3.14), or a retinol sulfotransferase/dehydratase;
or
E) (a) a sulfotransferase (EC 2.8.2); and
(b) an isopentenyl-diphosphate DELTA isomerase (EC 5.3.3.2); and
(c) a terpene synthase, an isoprene synthase (EC 4.2.3.27), a monoterpene synthase, an alpha-farnesene synthases (EC 4.2.3.46), an beta-farnesene synthase (EC 4.2.3.47), a myrcene/(E)-beta-ocimene synthase (EC 4.2.3.15), a pinene synthase (EC 4.2.3.14), or a retinol sulfotransferase/dehydratase.

30. A method of producing isoprene from isoprenol,
wherein the method comprises converting isoprenol into isoprene using an enzyme selected from:
(i) a terpene synthase, an isoprene synthase (EC 4.2.3.27), an alpha-farnesene synthases (EC 4.2.3.46), an beta-farnesene synthase (EC 4.2.3.47), a myrcene/(E)-beta-ocimene synthase (EC 4.2.3.15), or a pinene synthase (EC 4.2.3.14); or
(ii) a sulfotransferase (EC 2.8.2).
